(19) United States
(12) Patent Application Publication
(10) Pub. No.: US 2024/0150711 A1
(43) Pub. Date: May 9, 2024

(54) PERSONALIZED REDIRECTION AND REPROGRAMMING OF T CELLS FOR PRECISE TARGETING OF TUMORS

(71) Applicants: Dana-Farber Cancer Institute, Inc., Boston, MA (US); The General Hospital Corporation, Boston, MA (US)

(72) Inventors: Marc Schwartz, Boston, MA (US); Nir Hacohen, Boston, MA (US)

(73) Assignees: Dana-Farber Cancer Institute, Inc., Boston, MA (US); The General Hospital Corporation, Boston, MA (US)

(21) Appl. No.: 18/279,848

(22) PCT Filed: Mar. 1, 2022
(86) PCT No.: PCT/US2022/018389

§ 371 Date: Aug. 31, 2023

Related U.S. Application Data
(60) Provisional application No. 63/155,295, filed on Mar. 1, 2021.

(51) Int. Cl. C12N 5/0783; A61K 35/17; C12N 9/22; C12N 15/11; C12N 15/86

(52) U.S. Cl. CPC C12N 5/0636; A61K 35/17; C12N 9/22; C12N 15/11; C12N 15/86; C12N 2310/20; C12N 2501/60; C12N 2501/727; C12N 2740/15042; C12N 2740/15043

(57) ABSTRACT

The disclosure provides methods of reprogramming polyclonal T cells to maintain long-term persistence. The disclosure further provides methods of treatment, such as adoptive T cell transfer therapies, that harness Tscms for development of tumor-reactive T cells. In some embodiments, the disclosure provides methods and compositions for positive modulation of the Tscm-producing phenotype, e.g., positive modulation of TCF7 expression. Positive modulation of TCF7 expression allows for maintenance of an increased T number of stem-like T cells capable of both self-renewal and generation of differentiated, cytolytic progeny.

### BACKGROUND OF THE INVENTION

Adoptive T cell therapy using expanded tumor-infiltrating cells has been employed for decades to treat solid tumors, but challenges remain to make this a major therapeutic option for patients, including identification of tumor-specific T cells, reprogramming of T cells to be more effective and transfer of T cell receptors and other genetic material into T cells.

Despite the dramatic successes achieved with cellular therapy for B cell malignancies, translation of the same successes in solid tumors has achieved limited results, due to the intrinsic tumor diversity and lack of conserved tumor antigens that could be targeted by gene-modified lymphocytes. Adoptive transfer of polyclonal tumor-infiltrating lymphocytes (TILs) has been long-appreciated as a promising approach to controlling solid tumors. However, a major challenge to the consistent robustness of this strategy relies on the variable degree to which TIL products are comprised of T cells with tumor-specificity, and hence the identification of tumor-reactive T cells and the definition of their properties are desired. In addition, growing evidence has pointed to the highly dysfunctional states of tumor-infiltrating T cells, and hence, strategies to effectively re-activate the functionality of these cells to effectuate consistent tumor killing are needed.

### SUMMARY OF THE INVENTION

The present disclosure is concerned with providing T cells with stem-cell like (stem-like) properties through redirection and reprogramming of genetic expression patterns. The present disclosure provides methods of preparing modified T cells by modulating the expression in the cells of the TCF7 gene, which encodes the TCF7 transcription factor. The present disclosure also provides compositions for modulating TCF7 expression in T cells. The present disclosure further provides methods of genetic screening in a T cell or a population of T cells to identify regulators of TCF7 expression, such as positive regulators and negative regulators of TCF7 expression. The present disclosure further provides compositions and pharmaceutical compositions comprising modified T cells, including TCF7-modulated T cells. The present disclosure also provides methods of treatment of a subject by isolating T cells, modifying (or reprogamming) the T cells ex vivo, and administering the modified cells to the subject. In some embodiments, modifying the T cells comprises modulation of the expression of TCF7 in the cells, such as increasing TCF7 expression. Isolated T cells may be isolated from the subject. In some embodiments, the T cells may be further modified ex vivo through the genetic modification (or grafting) of one or more T cell receptors (TCRs) expressed by these T cells, such as TCRs possessing confirmed tumor-reactivity.

The present disclosure is based, at least in part, on the discovery that TCF7 expression is integral to long-term persistence of memory and stem-cell like T cells (Tscms). Tscms have the ability to differentiate into effector T cells, including those that are tumor-reactive. It is desired to generate populations of Tscms having long-term persistence in order to produce populations of differentiable and readily-expandable effector T cells that can recognize and neutralize tumor cells. The present disclosure is concerned with developing methods of treatment, such as adoptive T cell transfer therapies, that harness Tscms for development of tumor-reactive T cells. In particular, the disclosure is concerned with providing methods and compositions for positive modulation of the Tscm-producing phenotype, e.g., positive modulation of TCF7 expression. Positive modulation of TCF7 expression allows for reprogramming of the genetic expression patterns of polyclonal T cells to maintain persistence. It further allows for maintenance of an increased number of stem-like T cells capable of both self-renewal and generation of differentiated, cytolytic progeny. The present disclosure further provides methods for generating populations of tumor-specific stem-like T cells that mediate enhanced anti-tumor immunity and/or an improved response to immunotherapies such as anti-PD1 checkpoint inhibitor.

It was hypothesized that arming T lymphocytes with TCR specificities of polyclonal T cells possessing confirmed tumor-reactivity along with regulators promoting stem-cell memory functional state promotes effective tumor regression and would serve as an effective cell therapy for treating cancer. For instance, polyclonal T cells possessing confirmed tumor-reactivity along with regulators promoting stem-cell memory functional state may attack and kill blood cancers, and solid tumor tissue such as melanoma. As such, provided herein are methods to engineer and/or modify T cells to exhibit a stem-cell memory phenotype with greater persistence. Further provided herein are methods to expand antitumor T cells with a stem-cell memory phenotype, including engineered antitumor T cells. Further provided herein are methods for the modification and expansion of T cells expressing tumor-reactive TCRs on their surface by reprogramming these cells to have a stem-cell memory phenotype. In some embodiments, provided herein are methods of evaluating the ability of these T cells to kill tumors. In some embodiments, the tumor-reactive TCRs are associated with and/or isolated from tumor-infiltrating T cells (or TILs).

In some embodiments, a nucleic acid molecule encoding a tumor-reactive TCR is genetically inserted into the genome of some or all of the T cells. In some embodiments, the nucleic acid molecule is inserted by homology directed repair (or homologous recombination repair). In some embodiments, the nucleic acid molecule is inserted by a CRISPR-based system, such as a CRISPR-based homology directed repair system.

In some aspects, TCF7 expression may be modulated in naïve T cells using any of the methods described herein. Provided herein are methods of preparing a modified T cell comprising: i) isolating one or more naïve T cells; ii) contacting a T cell with an agent that stimulates expansion of the T cell; and iii) incubating the T cell with an inhibitor of a negative modulator gene of TCF7, e.g., for at least 18 hours. As used herein, “a negative modulator gene of TCF7” refers to a gene that, when expressed, acts to downregulate the expression of TCF7. In some embodiments, the T cell is incubated with the inhibitor for at least 18 hours, at least 24 hours, 30 hours, 36 hours, 48 hours, 54 hours, 72 hours, 4 days, or 5 days. In some embodiments, the negative modulator gene is JAK2 or STAT1. In some embodiments, the negative modulator gene is IFNGR1.

In some embodiments, the inhibitor is a small molecule inhibitor of the activity of the protein encoded by the negative modulator gene. In some embodiments, the inhibitor is a small molecule that associates with, or binds, the protein encoded by the negative modulator gene. In some embodiments, the inhibitor is ruxolitinib, baricitinib, fedratinib, gandotinib, lestaurtinib, momelotinib, pacritinib, pravastatin, ISS-840, fludarabine, OPB-31121, or heparin.

In some embodiments, the inhibitor is a biologic molecule. In some embodiments, the inhibitor is a nucleic acid molecule. In some embodiments, the inhibitor is a protein, such as an antibody or a cytokine. In some embodiments, the inhibitor is an RNA molecule, such as a short hairpin RNA or short interfering RNA. In some embodiments, the inhibitor is a short interfering RNA (siRNA) comprising a sequence of at least 10 contiguous nucleotides that is complementary to a portion of a genomic sequence comprising the negative modulator gene. In some embodiments, the inhibitor is a complex comprising i) a CRISPR-associated protein, and ii) a guide RNA comprising a guide sequence of at least 10 contiguous nucleotides that is complementary to a portion of a genomic sequence comprising the negative modulator gene. In some embodiments, the inhibitor is a polynucleotide encoding an siRNA molecule.

In some embodiments, the inhibitor is a polynucleotide encoding a complex comprising a CRISPR-associated protein and a guide RNA comprising a guide sequence of at least 10 contiguous nucleotides that is complementary to a portion of a genomic sequence comprising the negative modulator gene. In some embodiments, the CRISPR-associated protein is a fusion protein comprising a dCas9 domain and a second domain.

In some aspects, provided herein are methods of genetic screening of a T cell, such as a naïve T cell, for modulation of TCF7 expression. In some embodiments, these methods comprise i) evaluating a first level of expression of TCF7 protein in a naïve T cell; ii) contacting the T cell with a) a CRISPR-associated nuclease, and b) a guide RNA molecule that comprises a guide sequence of at least 10 contiguous nucleotides that is complementary to a target sequence in the genomic DNA of the T cell; iii) contacting the cell with an agent that stimulates expansion of the T cell; iv) evaluating a second level of expression of TCF7 protein in the T cell; and v) identifying the target sequence as a candidate modulator gene of TCF7 if the second level of expression exceeds the first level by more than 10%. In accordance with these methods, the target sequence is a sequence that encodes any candidate modulator gene. In some embodiments, the second level of expression exceeds the first level by more than 15%, 20%, 25%, 30%, or more than 30%. The step of contacting may be in vitro or in vivo. The step of contacting may be in vivo in an experimental animal subject.

In some embodiments, the candidate modulator gene is a gene that encodes a transcription factor. In some embodiments, the candidate modulator gene is a gene that encodes a Krüppel associated box (KRAB) domain. KRAB domains are transcriptional repression domains present in many zinc-finger proteins, such as zinc-finger nucleases (ZFNs).

Further provided herein are methods of genetic screening of a population of T cells, such as a population of naïve T cells. In some embodiments, these methods comprise: i) contacting a population of naïve T cells with a) a CRISPR-associated nuclease, and b) a library of guide RNA molecules, wherein each of the guide RNA molecules comprises a different guide sequence of at least 10 contiguous nucleotides that is complementary to a portion of a target sequence in the genomic DNA of a T cell; ii) contacting the population with an agent that stimulates expansion of the T cells in the population; iii) sorting the cells of the population based on level of expression of TCF7; iv) evaluating the abundance of any of the plurality of guide RNA molecules in the cells exhibiting substantially higher expression of TCF7; and v) identifying a target sequence as a candidate modulator gene if the abundance of the guide RNA molecule that is complementary to a portion of the candidate gene is substantially enriched in one or more cells exhibiting higher expression of TCF7. As used herein, the terms “substantially enriched” and “substantially higher expression” refer to an increase in expression of 10% or greater. In some embodiments, the step of contacting comprises transducing the one or more cells with one or more polynucleotides encoding the CRISPR-associated nuclease and the library of guide RNA molecules. In some embodiments, the one or more polynucleotides is comprised within one or more vectors, such as viral vectors, e.g., lentiviral vectors.

In some aspects, provided herein are lentiviral particles comprising a recombinant lentiviral vector comprising one or more polynucleotides encoding a) a CRISPR-associated nuclease, and b) a plurality of guide RNA molecules, wherein each of the guide RNA molecules comprises a different guide sequence of at least 10 contiguous nucleotides that is complementary to a target sequence in the genomic DNA of a T cell. In some embodiments, the lentiviral particle comprises VSV-g and ecotropic (ECO) envelope proteins.

In some aspects, provided herein are compositions of one or more modified T cells prepared according to any of the disclosed methods. In some embodiments, the T cells are polyclonal T cells. These polyclonal T cells may express a T-cell receptor (TCR) that is associated with a tumor-infiltrating lymphocyte (TIL). In some embodiments, these polyclonal T cells may express a chimeric antigen receptor (CAR), such as a CAR specific for a cancer antigen, such as CD19, CD123, CD133, and EGFR. In some embodiments, these polyclonal T cells are CAR-T cells. In various aspects, the T cells exhibit a memory or a stem-cell memory (Tscm) phenotype.

In some aspects, provided herein are pharmaceutical compositions or preparations of modified T cells comprising a pharmaceutically acceptable excipient. These compositions may be suitable for adoptive transfer to a subject. In some embodiments, these compositions may be adapted for autologous transfer to a subject. In some embodiments, these compositions may be adapted for allogeneic transfer to a subject. Provided herein are compositions, including pharmaceutical compositions, for use in treating cancer. In some embodiments, any of the disclosed compositions or preparations further comprise the administration of an immunotherapy, such as an FDA- or EMA-approved immunotherapy. In some embodiments, these compositions further comprise an immune checkpoint inhibitor such as an anti-PD1 inhibitor.

In other aspects, provided herein are methods of treatment. In some embodiments of these methods a subject suffering from, or diagnosed with, a cancer is treated by administering any of the disclosed compositions. In some aspects of the methods, provided are methods of treating a subject suffering from, or diagnosed with, a cancer comprising: i) isolating T cells from the blood of a subject; ii) contacting the T cells ex vivo with an inhibitor of a negative modulator gene of TCF7 for at least 18 hours, thereby producing modified T cells; and iii) administering the modified T cells to the subject. This comprises an autologous transfer or treatment. Also provided herein are methods of treatment comprising isolating the T cells from a first subject, contacting the T cells ex vivo as above, and administering the modified T cells to another subject. This comprises an allogeneic transfer or treatment. In particular embodiments, the subject is a human. The cancer may be a solid tumor. In other embodiments, the cancer is a blood cancer, such as a lymphoma or leukemia.

In some embodiments, the T cells express a T-cell receptor (TCR) that is associated with, or expressed by, a tumor-infiltrating lymphocyte (TIL). The modified T cells may exhibit a Tscm phenotype and/or TCF7 overexpression. The modified T cells may exhibit long-term persistence. In some embodiments, the modified T cells exhibit stem-like behavior and/or are resistant to T cell exhaustion. In some embodiments, the modified T cells exhibit stem-like behavior and are resistant to T cell exhaustion, and produce differentiated progeny T cells that do not exhibit stem-like behavior but are rather effector T cells. In some embodiments, the modified T cells produce progeny T cells that are tumor-reactive T cells. In some embodiments, these progeny T cells are cytolytic T cells, NK T cells, and/or CD8+ T cells. In some embodiments, these T cells are cytolytic CD8+ cells.

In some embodiments, the modified T cells are cytolytic T cells, NK T cells, and/or CD8+ T cells. In particular embodiments, the modified T cells are CD8+ T cells. In some embodiments, the modified T cells may express the cellular machinery to function in cytolytic killing of a tumor cell. In some embodiments the cells are peripheral blood lymphocytes. In some embodiments, the T cells express tumor-reactive TCRs and/or are used to assay cytolytic activity against subject specific tumor cells in vitro.

In some embodiments, the step of contacting comprises incubating the T cells with the inhibitor for at least 18 hours, 24 hours, 30 hours, 36 hours, 48 hours, 54 hours, 72 hours, 4 days, or 5 days. In particular embodiments, the step of contacting comprises incubating the T cells with the inhibitor for at least 24 hours. The isolating step may further comprise contacting the T cells with an agent that stimulates expansion of the T cells. The expansion-stimulating agent may comprise a vaccine. The expansion-stimulating agent may comprise an anti-CD3 antibody. The expansion-stimulating agent may comprise an anti-CD28 antibody. The expansion-stimulating agent may comprise a combination of an anti-CD3 antibody and an anti-CD28 antibody.

In some embodiments, any of the disclosed methods further comprise the administration of an immunotherapy, such as an FDA- or EMA-approved immunotherapy. In some embodiments, these methods further comprise the administration of immunotherapies an immune checkpoint inhibitor such as an anti-PD1 inhibitor.

In some aspects, provided herein are kits for the preparation of a medicament of modified T cells. In some aspects, provided herein are kits for isolating T cells from the blood of a subject, and contacting the T cells ex vivo with an inhibitor of a negative modulator gene of TCF7.

In some aspects, provided herein are preparations comprising any of the disclosed modified T cell compositions for use as a medicament to treat cancer. In some aspects, provided herein are preparations for use as a medicament to treat solid tumors.

### BRIEF DESCRIPTION OF THE DRAWINGS

FIGS. 1A-1C show that TCF7-expressing CD8+ T cells are critical for tumor clearance. FIG. 1A is a schematic that shows that TCF7+ cells correlate in their response to anti-PD1 inhibitors in a melanoma cell line. FIG. 1B shows the response of the mouse tumor cell line MC38 to checkpoint blockade immunotherapy (CBI), e.g., anti-PD1 antibody administration, depends on TCF7+ cells. FIG. 1C is a schematic showing TCF-mediated tumor cell death.

FIG. 2 shows the developed naïve CD8 T cell transduction method. Results show efficient transduction and Cas9-mediated deletion without altering surface phenotype. Data shown is from T cells 5 days after transduction with lentiviral single-guide RNA (sgRNA) vector.

FIGS. 3A and 3B show transduced T cells are functional in vivo. FIG. 3A shows the results of lentiviral transduction wherein P14 T cells were transferred to mice infected with acute lymphocytic choriomeningitis virus (LCMV), Armstrong strain. T cells were transduced, 5 days later transferred to recipient mice that were then infected with LCMV. Cells were analyzed at 7 and 30 days. FIG. 3B is an expression map that shows the tumor model wherein CD8+ T cells of OT1 mice (a C567/B mouse with a transgenic T cell receptor) were transferred to mice with tumors (the MC38 colon adenocarcinoma cell line).

FIG. 4 shows the protocol and fluorescence activated cell sorting (FACS) results following a “test” in vitro CRISPR genomic screen for TCF7 modulators (see Table 1). The depicted distribution indicates the gating strategy for the FACS sorting, which made use of a GFP fluorescent tag fused to TCF7 in the cells. Results show that the in vitro screen protocol effectively nominated known positive and negative regulator genes of TCF7, including negative regulators ID2, PRDM1, and RUNX3.

FIG. 5 shows FACS results of whole genome in vitro naïve CD8+ T cell CRISPR screens. The depicted distribution indicates the gating strategy for the FACS sorting. The screen identified genes that regulate TCF7 expression following CD8+ T cell activation/expansion, including negative regulators JAK2 and STAT1. These results provide a proof of concept of in vitro genome scale screens in naïve CD8+ T cells.

FIG. 6 shows the protocol and FACS results of an in vivo screen in a vaccine model. Results show that screen identified genes that control Tcf7 expression in vivo after CD8 T cells were activated by a vaccine.

FIGS. 7A-7C show the experimental setup and results of whole genome in vitro naïve CD8+ T cell CRISPR screens for Tcf7 modulators. FIG. 7A is a schematic that shows the experimental setup for in vitro naïve CD8 T cell screens to identify regulators of Tcf7. FIGS. 7B and 7C show the newly identified regulators of Tcf7. FIG. 7B is a volcano plot showing the gene targets of sgRNAs enriched in Tcf7 high cells (right side of plot) and Tcf7 low cells (left side of plot). The y-axis represents average p-values for each modulator gene. FIG. 7C shows a network analysis of gene ontology pathways enriched in all significant genes nominated by the Tcf7 screen, clustered based on overlapping genes. Adjusted p-values of each cluster of genes are shown at right-middle.

FIGS. 8A-8C show an in vitro system for T cell expansion. FIG. 8A shows a method for in vitro expansion of T cells that, without intervention, results in loss of Tcf7 expression in the majority of cells by 1 week post stimulation. FIG. 8B shows an analysis of Tcf7 protein levels in CD8 cells transduced with either control or IFN-gamma receptor 1 (Ifngr1)-targeting sgRNAs. FIG. 8C shows the quantification of this data at various time points post stimulation.

FIG. 9 shows the intracellular staining in OT1 mouse T cells of Tcf7 and IFN-γ two days after co-culture with B16-OVA tumor cells. The bottom right panel shows the number of live tumor cells quantified by flow cytometry for each well and compared to wells with no T cells present.

FIG. 10 shows quantification of viability of naïve CD8 T cells that received either control or IFN-γr1 (Ifngr1)Ifngr1-targeting sgRNAs were co-cultured with MC38-OVA tumor cells. The left panel shows number of live tumor cells in each well normalized to the average number of live tumor cells in wells without any T cells. The right panel shows the viability of T cells present in each well.

FIG. 11 is a schematic showing experimental method to evaluate the effect of blocking Ifn-gamma signaling in CD8+ T cells using a separate method from CRISPR-deletion.

FIG. 12 shows bar graphs showing the fraction of cells in each well that were positive for Tcf7 protein expression at day 5, day 7, day 8, and day 11 post stimulation in all conditions.

FIGS. 13A and 13B show results of an MC38-OVA co-culture with IFNGR antibody inhibition. FIG. 13A is a schematic showing naïve CD8 T cells were isolated from wild-type (WT) mice and treated with an isotype antibody or a combination of anti-Ifng and anti-IfngR neutralizing antibodies (NAbs). FIG. 13B shows number of live tumor cells in each well compared to wells without T cells (left) and viability of T cells present in each well (right) following MC38-OVA co-culture.

FIG. 14 is a schematic showing a general experimental setup for in vivo phenotyping experiments for inhibition of Ifngr1.

FIG. 15 shows the experimental setup for testing whether knockout of Ifngr1 provides a competitive advantage for CD8 T cells in vivo in the setting of either vaccination or tumors.

FIGS. 16A and 16B show an analysis of control and Ifngr1-sgRNA containing CD8 T cells 1 week after vaccination with OVA-CpG. The left panel demonstrates the starting ratio of control (double positive/DP) to Ifngr1 (CD45.1) that was initially injected into recipient mice. The top right panel shows the ratio 1 week after vaccination, showing a slight competitive advantage for IFngr1-sgRNA cells.

FIGS. 17A and 17B show an analysis of control and Ifngr1-sgRNA CD8 T cells isolated from tumor-draining lymph nodes (dLN) 1 week after implant of B16-OVA tumors.

FIGS. 18A and 18B show an analysis of control and Ifngr1-sgRNA CD8 T cells isolated from tumor-draining lymph nodes (dLN) 13 days after implant of B16-OVA tumors, and 5 days after treatment with either isotype of anti-PD1 antibodies.

FIGS. 19A and 19B show an analysis of control and Ifngr1-sgRNA CD8 T cells isolated from tumor-draining lymph nodes (dLN) 13 days after implant of B16-OVA tumors, and 5 days after treatment with either isotype of anti-PD1 antibodies. The bottom left panel highlights persistence of a less-activated, CD44-low T cell population in Ifngr1-deficient T cells only.

FIGS. 20A and 20B show an analysis of control and Ifngr1-sgRNA CD8 T cells isolated directly from tumors 13 days after implant of B16-OVA tumors, and 5 days after treatment with either isotype of anti-PD1 antibodies.

FIG. 21 is a schematic showing the evaluation of the effects of Ifngr1 deletion on T cell persistence in vivo, following delivery of non-targeting or Ifngr1-sgRNAs were to naïve CD8 T cells isolated from Cas9/OT1-double transgenic mice.

FIGS. 22A and 22B show that using congenic markers CD45.1 and CD45.2, transferred OT1 cells were only detected in mice that received Ifngr1-sgRNA containing CD8 T cells.

### DETAILED DESCRIPTION OF THE INVENTION

Challenges remain in the art to make adoptive T cell therapy of tumor infiltrating lymphocytes (TILs) a major therapeutic option for patients, including the reprogramming of T cells to be more effective. The present disclosure addresses this reprogramming challenge, through which fresh insights have been gained in recent studies. This disclosure seeks to provide patients with effective and functional T cell immunity against cancer cells.

Through the intensive dissection of the T cell repertoire of lymphocytes infiltrating melanoma tissue, previous studies have evaluated the links among antigen specificity, TCR avidity and T cell functional states and have identified that tumor tissue is in fact a source of polyclonal T cells with exquisite tumor specificity, but at baseline the functional state of these polyclonal T cells is primarily that of exhaustion. The disclosed experimental results dovetail closely with other studies which documented the intratumoral presence of CD8+ T cells expressing high levels of co-inhibitor genes (e.g., CD39, TIM-3, PD1) and their association with poor response to immunotherapy in patients with melanoma. PD1 is an immune checkpoint protein that may be of particular relevance to immunotherapy outcomes. Conversely, several recent studies have shown that tumor regression is often associated with the presence of antitumor T cells with stem-cell like properties that are most effective at eliminating tumors because of their ability to provide a renewable source of effector T cells. These antitumor T cells with stem-cell like properties retain a memory phenotype and avoid exhaustion. Therefore, the identification of regulators of this cell state could pave the road to the ex vivo reprogramming and expansion of T cells with antitumor TCRs towards a beneficial memory phenotype. The ability to surgically substitute the endogenous TCR with a new antitumor specificity, coupled with the insertion of known T-cell costimulatory factors, has been demonstrated.

Cellular therapy has been transformative for the treatment of cancer, with the most dramatic results observed for B cell malignancies. A longstanding goal for cancer treatment has been the harnessing of the specificity and potency of T lymphocytes for effective cytotoxicity against cancer. Over the past decade, the advent and FDA approvals of chimeric antigen-receptor (CAR) T cells for the treatment of CD19+ B cell malignancies have provided a clear demonstration of the potency of cellular therapy for the treatment of cancers. For solid tumors, however, major barriers include identifying targets and the engineering and reprogramming of T cells for effective entry into tumors, activation and persistence, and subsequent complete elimination of tumors. Tumor infiltrating lymphocyte (TIL)-based therapies have been used for many years for the treatment of solid tumors. It has been long appreciated that tumor-reactive T cells are found in tumor infiltrates and can be used for adoptive T cell transfer therapy through decades of clinical experience in collecting, expanding and infusing TILs against solid cancers. The variability in clinical activity, however, is due to the frequency of tumor-reactive TILs and the level of function/dysfunction of the T cells. More effective ways to select tumor-reactive T cells and maximize their activity must be developed to make adoptive T cell therapy a routine and effective therapy.

Several genetic screening technologies have been developed. For example, single cell RNA sequencing was used to detect populations of T cells with specific phenotypes (Sade-Feldman et al. 2018, Cell, 175(4): 998-1013), and the isolation and cloning of T cell receptors (Hu et al. Blood, 2018 November; 132(18): 1911-1921) allowed rapid identification of numerous tumor-reactive TCRs based on surface markers on tumor-infiltrating T cells (Noviello et al., Nature Communications 10(1065) (2019); see also Oliveira et al. Nature 2021; 596(7870):119-125, and Hu, Ott & Wu, Nature Rev. Immunol. 18, 168-182 (2018)), each of which is incorporated herein by reference. In addition, large-scale pooled CRISPR screens have been used to identify genes involved in controlling T cell fates, enabling the improved manipulation of T cell stem-cell activities.

Key current challenges in cancer therapeutics include how to address the clonal heterogeneity of tumors, and how to treat cancers in a personalized fashion since abundant past experiences suggest that a ‘one size fits all’ approach rarely provides long-term curative therapy for many solid tumor malignancies. Indeed, for solid tumors, solitary targetable surface antigens that are tumor-restricted are rare. As described herein, new approaches for (i) parallel identification of multiple tumor-reactive TCRs per patient, (ii) generation of optimal T cell state for adoptive cellular therapy, such as the Tscm state are described herein.

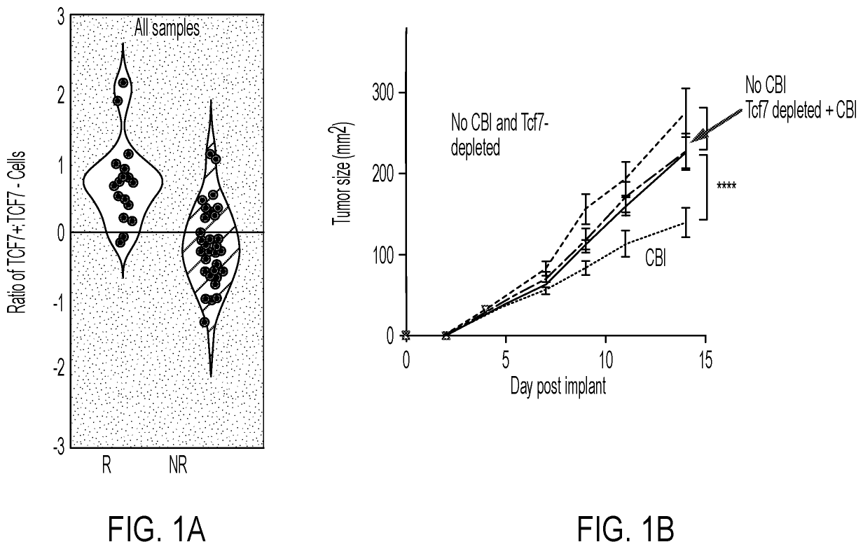

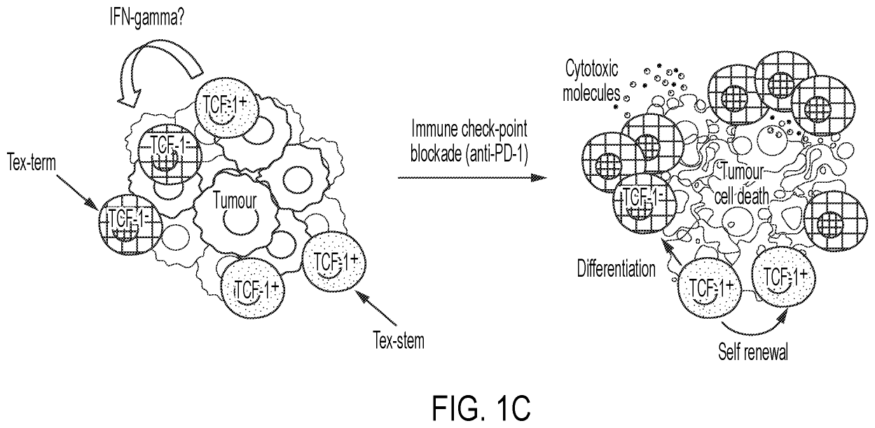

The TCF7 gene encodes the TCF7 transcription factor (which is also known as TCF-1 transcription factor) (see FIG. 1C). TCF7 is important for T cell development and differentiation. It is believed that the activity of this transcription factor promotes the memory stem cell phenotype of T cells. Furthermore, Krishna et al. recently demonstrated that adoptive T cell immunotherapies that contan high numbers of TCF7-expressing TILs exhibit superior self-renewal, expansion, persistence, and activity against tumors. See Krishna et al., Science 2020 December; 370(6522):1328-1334, which is incorporated herein by reference. In particular, this group determined that the CD39-negative stem-like phenotype (CD39−CD69−) was associated with TIL persistence and expansion. It is thus hypothesized that inhibition of a negative modulator gene of TCF7—and contrapositively, the activation or promotion of a positive modulator gene of TCF7—will enhance the generation of TCF7-expressing (TCF7+) cells, promote TCF7 activity, and/or promote the memory stem cell phenotype.

In some aspects, provided herein are methods of modification of naïve T cells to produce a positive modulation of TCF7. Also provided herein are methods of modification of naïve T cells to inhibit a negative modulation of TCF7. In some embodiments, the candidate modulator gene is a negative modulator gene, such as a gene that silences the activity of TCF7.

Provided herein are methods of preparing a population of modified T cells. In some embodiments, the methods are performed in vitro or ex vivo. In some embodiments, the modified T cells are modified primary T cells. In some embodiments, they are modified naïve T cells. In some embodiments, the modified T cells are TILs. In some embodiments, the modified T cells are CD8+T cells. In some embodiments, the modified T cells are isolated from a subject, such as a human subject. In some embodiments, the modified T cells have been modified from exhibiting a phenotype of exhaustion to one of stem-like persistence.

In some embodiments, the candidate modulator gene is a negative modulator of TCF7. In some embodiments, the candidate modulator gene is a positive modulator of TCF7. The candidate modulator gene may be selected from the following genes: Actb, Hif1a, Ndufs2, Samd1, Apim1, Hira, Nelfb, Slc2a1, Arhgap1, Hnrnpab, Pabpc1, Slc35a2, Arnt, IFN-γR1 (Ifngr1), Pgd, Sic7a1, Atxn7l3, IFN-γR2 (Ifngr2), Pgk1, Sic7a5, Bap1, Il21r, Pgm3, Smarca4, Brd4, Il2ra, Phip, Smarce1, Ccdc6, Ints5, Pik3cd, Socs3, Chd4, Ints6, Pik3cg, Spcs2, Cited2, Irf4, Pik3r5, Spcs3, Cox7b, Itk, Pkm, Stat1, Ctcf, Jak2, Ppp2r2a, Stat3, Dcun1d3, Lck, Prdm1, Stk11, Dnmt1, Ldha, Pten, Sumo2, Dnttip1, Mapk14, Ptpn1, Tcf7, Drosha, Mbd2, Ptpn2, Tfap4, Dyrk1a, Med13, Ptpn6, Tkt, Elovl1, Med16, Ptprc, Traf3, Emc6, Mideas, Rack1, Ube2m, Fbxo42, Mist8, Rbbp4, Uhrf1, Foxo1, Mob4, Rc3h1, Umps, Gale, Myb, Rp128, Vhl, Girx5, Naa20, Rpl28-ps1, Washc4, Gmppb, Ndrg3, Rtf2, Zc3h12a, Gpi1, Ndufa6, Runx3, and Zfp384. The candidate modulator gene may be selected from the following genes: Actb, Arnt, Asf1a, Atxn713, Cnot3, Ctcf, Dctn5, Ddx11, Dnajc11, Dyrk1a, Fbxo42, Girx5, Gpi1, Hif1a, Hira, Ifngr1, Ifngr2, Irf1, Jak2, Kif20a, Ldha, Lsm10, Mideas, Mrp153, Pkm, Ppp2r2a, Prdm1, Rnaseh2a, Runx3, Samd1, Smarce1, Smc4, Spcs2, Spcs3, Stat1, Stat3, Tbx2, Tfg, and Triml2. The candidate modulator gene may be selected from a gene listed in Table 1.

In some embodiments, the candidate modulator gene is in a gene selected from the group consisting of IFNGR1, Jak2, Arnt, Stat1, Irf1, Tbx2, Tfg, Ppp2r2a, Dctn5, Asf1a, Ddx11, Hira, Smc4, Mrp153, Triml2, and Dnajc11. The candidate modulator gene may be the JAK2 gene. The candidate modulator gene may be the STAT1 gene. The candidate modulator gene may be the IFNGR1 gene. All of IFNGR1, Jak2, Arnt, Stat1, Irf1, Tbx2, Tfg, Ppp2r2a, Dctn5, Asf1a, Ddx11, Hira, Smc4, Mrp153, Triml2, and Dnajc11 are believed to be negative modulators of TCF7.

In some embodiments, the candidate modulator gene is Ldha, which encodes the major isoform of lactate dehydrogenase (LDH). A recent mouse study suggested the importance of Ldha in self-renewal of antitutmor TILs. See Hermans et al., PNAS 2020 Mar. 17; 117(11):6047-6055, which is incorporated herein by reference. In this study, Ldha inhibition combined with IL-21 increased the formation of Tscm cells, resulting in stronger antitumor responses.

In some embodiments, the candidate modulator gene is Ifngr1 or Ifngr2. In some embodiments, the candidate modulator gene is Ifngr1.

Provided herein are methods of preparing a population of modified T cells comprising: i) isolating a population of naïve T cells; ii) contacting the population with an agent that stimulates expansion of the T cells; and iii) incubating the T cells with an inhibitor of a negative modulator gene of TCF7 for at least 18 hours. In some embodiments, the T cells exhibit a memory or a stem-cell memory (Tscm) phenotype after the step of incubating.

The methods may further comprise the step of iv) engineering the T cell or T cells to express an antigen-specific T-cell receptor (TCR) that is associated with a tumor-infiltrating lymphocyte (TIL) in a subject, e.g., using homology directed repair. The methods may further comprise v) contacting the T cell or T cells with one or more T cell costimulatory factors.

Further provided herein are methods of modifying the T cells of a subject comprising administering to the subject: i) an agent that stimulates expansion of the subject's T cells; and ii) an inhibitor of a negative modulator gene of TCF7. In some embodiments, an amount of the inhibitor is administered that is effective to promote TCF7 activity in the subject's T cells. In some embodiments, the subject's T cells exhibit a memory and/or a stem-cell memory (Tscm) phenotype. In some embodiments, the subject is a human.

In some embodiments, the inhibitor is a small molecule inhibitor of JAK2, such as an inhibitor is selected from ruxolitinib, baricitinib, fedratinib, gandotinib, lestaurtinib, momelotinib, and pacritinib. In other embodiments, the inhibitor is a small molecule inhibitor of STAT1, such as an inhibitor selected from pravastatin, ISS-840, fludarabine, and OPB-31121. Additional inhibitors of the JAK-STAT pathway, and in turn JAK2 and/or STAT1, are disclosed in US Patent Pub. No. 2015/0157597, published Jun. 11, 2015. In some embodiments, the inhibitor is a small molecule inhibitor of a gene encoding an interferon-gamma receptor, such as IFNGR1. Exemplary inhibitors of the activity of IFNGR1 and/or the IFN-γ receptor include heparin and IL-10. These inhibitors are described in Hatakeyama et al., “Heparin Inhibits IFN-Gamma-Induced Fractalkine/CX3CL1 Expression in Human Endothelial Cells,” Inflammation 28(1):7-13 (2004) and Song et al., “Interleukin-10 Inhibits Interferon-Gamma-Induced Intercellular Adhesion Molecule-1 Gene Transcription in Human Monocytes,” Blood 89(12):4461-9 (1997), which are hereby incorporated by reference in their entirety. Additional IFNGR inhibitors are described in US Patent Pub. No. 2013/0142809, published Jun. 6, 2013. The inhibitors for use in accordance with the disclosed methods and compositions are not limited to those listed above.

In some embodiments, the inhibitor is a small molecule inhibitor of Arnt, Irf1, Tbx2, Tfg, Ppp2r2a, Dctn5, Asf1a, Ddx11, Hira, Smc4, Mrp153, Triml2, or Dnajc11. In some embodiments, the inhibitor is an siRNA comprising a sequence of at least 10 contiguous nucleotides that is complementary to a portion of a genomic sequence comprising Jak2, Stat1, Arnt, Irf1, Tbx2, Tfg, Ppp2r2a, Dctn5, Asf1a, Ddx11, Hira, Smc4, Mrp153, Triml2, or Dnajc11.

In some embodiments, the step of isolating comprises isolating and/or purifying the T cells from the blood of a subject. The step of isolating may comprise isolating and/or purifying the T cells from the blood of a cancer patient.

### Definitions

As used herein and in the claims, the singular forms “a,” “an,” and “the” include the singular and the plural reference unless the context clearly indicates otherwise. Thus, for example, a reference to “an agent” includes a single agent and a plurality of such agents.

As used herein, the terms “negative modulator gene” refers to a gene that regulates the expression of a second gene such that when expressed or activated, the product of the gene downregulates expression or activity of the second gene. Generally, a negative modulator is positioned higher in a cellular signal-transduction pathway than the second gene, or the modulated gene. In some embodiments, the negative modulator gene is a transcription factor. The modulated gene may also be a transcription factor. In various embodiments of the disclosure, a “negative modulator gene of TCF7” refers to a gene (e.g., STAT1, JAK2) that, when expressed, acts to downregulate the expression of TCF7 such that less TCF7 protein is produced. A “negative modulator protein” may refer to the protein product of a negative modulator gene. Negative modulator genes may be novel or known a priori.

As used herein, the term “candidate modulator gene” refers to a gene that has not been previously identified as a negative modulator gene (e.g., modulator of TCF7 expression) but is subject to a screen that may subsequently identify it as a negative modulator gene. Candidate modulator genes are typically not known by the experimenter a priori to be negative modulator genes.

As used herein, the term “cancer” may refer to any cancer, including any of sarcomas (e.g., synovial sarcoma, osteogenic sarcoma, leiomyosarcoma uteri, and alveolar rhabdomyosarcoma), lymphomas (e.g., Hodgkin lymphoma and non-Hodgkin lymphoma), hepatocellular carcinoma, glioma, head-neck cancer, acute lymphocytic cancer, acute myeloid leukemia, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer (e.g., colon carcinoma), esophageal cancer, cervical cancer, gastrointestinal cancer (e.g., gastrointestinal carcinoid tumor), hypopharynx cancer, larynx cancer, liver cancer, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, rectal cancer, renal cancer, small intestine cancer, soft tissue cancer, stomach cancer, testicular cancer, thyroid cancer, ureter cancer, and urinary bladder cancer. In some embodiments, the cancer is a solid tumor.

As used herein, the term “vaccine” refers to a composition for generating immunity, e.g., a medicament that comprises antigens and are intended to be used in humans or animals for generating specific defence and protective substance by vaccination. In some embodiments, the vaccines of the disclosure refer to medicaments comprising an antigen intended to be used in an experimental animal, in a genomic CRISPR screen. In some embodiments, the vaccine of the disclosure is OVA-CpG.

By “polynucleotide” is meant a nucleic acid (e.g., a DNA) that is free of the genes which, in the naturally-occurring genome of the organism—or in the genomic DNA of a T cell isolated from the organism—the nucleic acid molecule of the disclosure is derived. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

As used herein, the terms “substantially enriched” and “substantially higher expression” refer to an increase in expression or abundance of an RNA molecule, or expression of a protein, of 10% or greater. For instance, a substantial enrichment in abundance of guide RNA molecules may involve a 10% or greater increase in abundance of those molecules at one or more timepoints of interest. Substantial enrichment and/or substantial increase in expression may involve a 15%, 20%, 25%, 30%, 35%, or greater than 35% increase in abundance or expression.

The term “patient” or “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline. In some embodiments, the subject is an experimental subject, such as an experimental animal, such as a rodent. In some embodiments, the subject is a human.

“Pharmaceutically acceptable” refers to approved or approvable by a regulatory agency of the Federal or a state government or listed in the U.S. Pharmacopeia, European Pharmacopoeia, or another generally recognized pharmacopeia for use in animals, including humans.

“Pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

The term “tumor reactivity” refers to activity against a tumor cell antigen. In some embodiments, the tumor cell antigen is CD19. In some embodiments, the tumor cell antigen is EGFR. In some embodiments, the tumor cell antigen is CD123 or CD133. The term “tumor-reactive T cell” refers to a T cell with special affinity for a tumor antigen and whose function is restricted to a tumor

As used herein, the terms “adoptive transfer,” “adoptive immunotherapy,” and “adoptive cell therapy” refer to the transfer of cells, most commonly immune-derived cells such as T cells, into the same patient or into a new recipient host with the goal of transferring the immunologic functionality and characteristics into the new host. When transfer is into the same patient, it is referred to as “autologous therapy.” When transfer is from one patient into another patient, it is referred to as “allogeneic therapy.”

### Methods of Genetic Screening

In some aspects, provided herein are methods of genetic screening of a population of T cells, e.g., for increased expression of TCF7. In some embodiments, these methods comprise genomic screening that comprises the transduction into naïve T cells (e.g., naïve CD8+ T cells) of one or more polynucleotides that are comprised within one or more viral vectors. In some embodiments, these methods comprise genomic CRISPR screening that comprises the transduction into naïve T cells (e.g., naïve CD8+ T cells) of one or more polynucleotides that are comprised within one or more lentiviral vectors. In some embodiments, the methods of genetic screening are performed in vitro. In some embodiments, the methods of genetic screening are performed in vivo. In some embodiments, the methods of genetic screening are whole genome in vitro screening methods.

Provided herein are methods of genetic screening of a population of T cells. In some embodiments, these methods comprise: i) contacting a population of naïve T cells with a) a CRISPR-associated nuclease, and b) a library of guide RNA molecules, wherein each of the guide RNA molecules comprises a different guide sequence of at least 10 contiguous nucleotides that is complementary to a portion of a target sequence in the genomic DNA of a T cell; ii) contacting the population with an agent that stimulates expansion of the T cells in the population; iii) sorting the cells of the population based on level of expression of TCF7; iv) evaluating the abundance of any of the plurality of guide RNA molecules in the cells exhibiting substantially higher expression of TCF7; and v) identifying a target sequence as a candidate modulator gene if the abundance of the guide RNA molecule that is complementary to a portion of the candidate gene is substantially enriched in one or more cells exhibiting higher expression of TCF7. In accordance with these methods, the target sequence is a sequence that encodes any candidate modulator gene. In some embodiments, the step of contacting comprises transducing the one or more cells with one or more polynucleotides encoding the CRISPR-associated nuclease and the library of guide RNA molecules.

In some embodiments, the one or more polynucleotides is comprised within one or more lentiviral vectors. In some embodiments, each of the lentiviral vectors is encapsulated in a lentiviral envelope. In some embodiments, the lentiviral envelope comprises VSV-g and ecotropic envelope proteins.

In some embodiments, the CRISPR-associated nuclease is a Cas9 nuclease. The Cas9 nuclease may be derived from any species. In some embodiments, the Cas9 nuclease is derived from S. pyogenes or S. aureus. In particular embodiments, the CRISPR-associated nuclease is an S. pyogenes Cas9.

In some embodiments, each of the guide RNA molecules is a single-guide RNA (sgRNA) molecule. In some embodiments, the library comprises at least 10, at least 15, at least 20, at least 30, at least 40, at least 50, at least 60, at least 65, at least 70, at least 75, at least 80, at least 90, or at least 100 guide RNA molecules.

In some embodiments of these screening methods, the candidate modulator gene is a human gene. In some embodiments of these screening methods, the candidate modulator gene encodes a transcription factor. The candidate modulator gene may be selected from Actb, Arnt, Asf1a, Atxn713, Cnot3, Ctcf, Dctn5, Ddx11, Dnajc11, Dyrk1a, Fbxo42, Glrx5, Gpi1, Hif1a, Hira, Ifngr1, Ifngr2, Irf1, Jak2, Kif20a, Ldha, Lsm10, Mideas, Mrp153, Pkm, Ppp2r2a, Prdm1, Rnaseh2a, Runx3, Samd1, Smarce1, Smc4, Spcs2, Spcs3, Stat1, Stat3, Tbx2, Tfg, and Triml2. The candidate modulator gene may be a gene selected from the sub-group consisting of JAK2, ARNT, STAT1, IRF1, TBX2, TFG, PPP2R2A, DCTN5, ASF1A, DDX11, HIRA, SMC4, MRPL53, TRIML2, and DNAJC1l. The candidate modulator gene may be the JAK2 gene. The candidate modulator gene may be the STAT1 gene. The candidate modulator gene may be the IFNGR1 gene.

In some embodiments, the agent that stimulates expansion of the T cell comprises a monoclonal anti-CD3 antibody and/or a monoclonal anti-CD28 antibody. In some embodiments, the step of sorting comprises flow cytometry-mediated sorting based on expression of a fluorescent reporter gene that is co-expressed with TCF7 in the cell.

In some embodiments, these methods further comprising: vi) administering to a tissue of interest in an experimental subject a population of T cells comprising i) a plurality of guide RNA molecules, wherein each of the guide RNA molecules comprises a different guide sequence of at least 10 contiguous nucleotides that is complementary to a portion of a candidate modulator gene, and ii) a CRISPR-associated protein; vii) administering to the tissue of interest an agent that stimulates T cell expansion; viii) evaluating the abundance of any of the plurality of guide RNA molecules in the tissue of interest at one or more time points; and ix) identifying a candidate modulator gene as a negative modulator gene of TCF7 if the abundance of a guide RNA molecule comprising a guide sequence that is complementary to the candidate modulator gene is substantially enriched in the population over time. As used herein, “substantially enriched” may involve a 10% or greater increase in abundance. Substantial enrichment may involve a 15%, 20%, 25%, 30%, 35%, or greater than 35% increase in abundance. Step viii) may further comprise evaluating the degree of proliferation of one or more T cells in the tissue of interest. The experimental subject may be a rodent.

In some embodiments, the CRISPR-associated protein comprises a nuclease-inactive Cas9 (dcas9) protein. In some embodiments, each of the guide molecules is a doxycycline-inducible sgRNA molecule. In some embodiments, the tissue of interest is tumor tissue or lymphatic tissue.

In particular embodiments, tissue of interest is tumor tissue. In some embodiments, the agent that stimulates T cell expansion is a vaccine. Step ix) may further comprise evaluating changes in size of the tumor tissue over time. The transcription factor may be a KRAB zinc finger protein.

The plurality of guide RNA molecules may comprise at least 10, at least 15, at least 20, at least 30, at least 35, at least 40, at least 45, or at least 50 guide RNA molecules. In some embodiments, the CRISPR-associated protein comprises a fusion of a dCas9 protein and a transcription factor. The cells in the tumorigenic tissue may exhibit surface expression of ovalbumin (OVA) antigen. In some embodiments, the method is performed in vitro or ex vivo.

Further provided herein are methods of genetic screening comprising: contacting a population of naïve T cells with one or more lentiviral particles containing a recombinant vector comprising polynucleotides encoding a) a CRISPR-associated nuclease, and b) a plurality of guide RNA molecules, wherein each of the guide RNA molecules comprises a different guide sequence of at least 10 contiguous nucleotides that is complementary to a target sequence in the genomic DNA of a T cell, whereby the population of cells are transduced with the plurality of guide RNA molecules, and wherein the one or more lentiviral particles comprise VSV-g and ecotropic envelope proteins.

In some embodiments, at least 60%, at least 65%, at least 70%, at least 75%, at least 80%, at least 85%, at least 90%, at least 95%, or at least 99% of the T cells are viable 72 hours after contacting with the one or more lentiviral particles. Following the step of contacting, the T cells exhibit a memory or a stem-cell memory (Tscm) phenotype. The method may further comprise contacting the population with one or more the reagents selected from RetroNectin®, LentiBOOST P®, recombinant IL-7, and recombinant IL-15.

### Vectors

Among vectors that may be used in the practice of the invention, integration in the host genome of a T cell is possible with retrovirus gene transfer methods, often resulting in long term expression of the inserted transgene. In certain embodiments, the retrovirus is a lentivirus.

Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Additionally, cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers.

Exemplary lentiviruses for the methods of transduction disclosed herein comprise a lentival particle comprising a lentiviral vector. The lentiviral particles may comprise two or more envelop proteins. The lentiviral particles may comprise a VSV-G envelope protein and an ecotropic, or ECO (a mouse specific envelope protein from the murine leukemia virus) envelope protein. The lentiviral particles may comprise additional envelope proteins.

In exemplary transduction protocols of the disclosure, the lentiviral particles contain VSV-g and ecotropic envelope proteins. Examples of ecotropic proteins include ECO and MLV. Chiefly, the protocol comprises the steps of packaging lentivirus with VSV-g and ECO envelopes, spinning virus onto retronectin coated plates, isolating naïve CD8s from mice immediately before the transduction, culturing in IL-7 and IL-15, and using LentiBOOST as a transduction enhancer.

### Methods of Treatment

In some embodiments, adoptive transfer therapies are contemplated herein. In some embodiments, methods of treatment comprise isolating T cells from a subject, contacting ex vivo the naïve T cells of a subject using any of the methods of preparing a modified T cells described herein, and infusing the modified T cells into the same subject or another subject. This type of treatment is referred to herein as an adoptive transfer therapy. Adoptive transfer therapies of the disclosure may be autologous or allogeneic.

Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73).

Ins some embodiments, T cells are expanded using the methods described herein and methods known in the art. Expanded T cells that express tumor specific TCRs may be administered back to a subject. In other embodiments, PBMCs are transduced or transfected with polynucleotides for expression of TCRs and administered to a subject. T cells expressing TCRs specific to neoantigens are expanded and administered back to a subject. In some embodiments, T cells that express TCRs that result in cytolytic activity when incubated with autologous tumor tissue are expanded and administered to a subject. In some embodiments, T cells that express TCRs that when used in the functional assays described herein result in binding to neoantigens are expanded and administered to a subject. In other embodiments, TCRs that have been determined to bind to subject specific neoantigens are expressed in T cells and administered to a subject.

In certain aspects, provided are methods of treating a subject suffering from, or diagnosed with, a cancer comprising: i) isolating T cells from the blood of a subject; ii) contacting the T cells ex vivo with an inhibitor of a negative modulator gene of TCF7 for at least 18 hours, thereby producing modified T cells; and iii) administering the modified T cells to the subject. This comprises an autologous transfer or treatment. Also provided herein are methods of treatment comprising isolating the T cells from a first subject, contacting the T cells ex vivo, and administering the modified T cells to another subject. This comprises an allogeneic transfer or treatment. In particular embodiments, the subject is a human. The cancer may be a solid tumor. In other embodiments, the cancer is a blood cancer, such as a lymphoma or leukemia.

In some embodiments, the T cells express a T-cell receptor (TCR) that is associated with, or expressed by, a tumor-infiltrating lymphocyte (TIL). The modified T cells may exhibit a Tscm phenotype and/or TCF7 overexpression. The modified T cells may exhibit long-term persistence. In some embodiments, the modified T cells exhibit stem-like behavior and/or are resistant to T cell exhaustion. In some embodiments, the modified T cells exhibit stem-like behavior and are resistant to T cell exhaustion, and produce differentiated progeny T cells that do not exhibit stem-like behavior but are rather effector T cells. In some embodiments, the modified T cells produce progeny T cells that are tumor-reactive T cells. In some embodiments, these progeny T cells are cytolytic T cells, NK T cells, and/or CD8+ T cells. In some embodiments, these T cells are cytolytic CD8+ cells.

In some embodiments, the modified T cells are cytolytic T cells, NK T cells, and/or CD8+ T cells. In particular embodiments, the modified T cells are CD8+ T cells. In some embodiments, the modified T cells may express the cellular machinery to function in cytolytic killing of a tumor cell. In some embodiments the cells are peripheral blood lymphocytes. In some embodiments, the T cells express tumor-reactive TCRs and/or are used to assay cytolytic activity against subject specific tumor cells in vitro.

In some embodiments, the step of contacting comprises incubating the T cells with the inhibitor for at least 18 hours, 24 hours, 30 hours, 36 hours, 48 hours, 54 hours, 72 hours, 4 days, or 5 days. In some embodiments, the step of contacting involves incubation for 18 hours. In some embodiments, the step of contacting involves incubation for 18-24, 18-30, 18-36, 18-48, 18-54, or 18-72 hours. In particular embodiments, the step of contacting comprises incubating the T cells with the inhibitor for at least 24 hours. The isolating step may further comprise contacting the T cells with an agent that stimulates expansion of the T cells. The expansion-stimulating agent may comprise a vaccine. The expansion-stimulating agent may comprise an anti-CD3 antibody. The expansion-stimulating agent may comprise an anti-CD28 antibody. The expansion-stimulating agent may comprise a combination of an anti-CD3 antibody and an anti-CD28 antibody.

In some embodiments, any of the disclosed methods further comprise the administration of an immunotherapy, such as an FDA- or EMA-approved immunotherapy. In some embodiments, these methods further comprise the administration of immunotherapies an immune checkpoint inhibitor such as an anti-PD1 inhibitor.

Aspects of the invention involve the adoptive transfer of immune system cells, such as T cells, specific for selected antigens, such as tumor associated antigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; and, Restifo et al., 2015, Adoptive immunotherapy for cancer: harnessing the T cell response. Nat. Rev. Immunol. 12(4): 269-281). Various strategies may for example be employed to genetically modify T cells by altering the specificity of the T cell receptor (TCR) for example by introducing new TCR a and R chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO20051 13595, WO2006125962, WO2013 166321, WO2013039889, WO2014018863, WO2014083173; U.S. Pat. No. 8,088,379).

Cell therapy methods often involve ex vivo activation and expansion of T-cells. In one embodiment T cells are activated before administering them to a subject in need thereof. Activation or stimulation methods have been described herein and is preferably required before T cells are administered to a subject in need thereof. Examples of these type of treatments include the use tumor infiltrating lymphocyte (TIL) cells (see U.S. Pat. No. 5,126,132), cytotoxic T-cells (see U.S. Pat. Nos. 6,255,073; and 5,846,827), expanded tumor draining lymph node cells (see U.S. Pat. No. 6,251,385), such as inguinal (groin) lymph nodes, and various other lymphocyte preparations (see U.S. Pat. Nos. 6,194,207; 5,443,983; 6,040,177; and 5,766,920). These patents are herein incorporated by reference in their entirety.

For maximum effectiveness of T-cells in cell therapy protocols, the ex vivo activated T-cell populations should be in a state that can maximally orchestrate an immune response to cancer, infectious diseases, or other disease states. For an effective T-cell response, the T-cells first must be activated. For activation, at least two signals are required to be delivered to the Tcells. The first signal is normally delivered through the T-cell receptor (TCR) on the T-cell surface. The TCR first signal is normally triggered upon interaction of the TCR with peptide antigens expressed in conjunction with an MHC complex on the surface of an antigen-presenting cell (APC). The second signal is normally delivered through co-stimulatory receptors on the surface of T-cells. Co-stimulatory receptors are generally triggered by corresponding ligands or cytokines expressed on the surface of APCs.

It is contemplated that the T cells obtained by the inventive methods can be used in methods of treating or preventing cancer. In this regard, the invention provides a method of treating or preventing cancer in a subject, comprising administering to the subject the pharmaceutical compositions or cell populations obtained by any of the methods described herein in an amount effective to treat or prevent cancer in the subject. Another embodiment of the invention provides a method of treating or preventing cancer in a subject, comprising administering a cell population enriched for tumor-reactive T cells to a subject by any of the inventive methods described herein in an amount effective to treat or prevent cancer in the mammal.

For purposes of the inventive methods, wherein populations of cells are administered, the cells can be cells that are allogeneic or autologous to the subject. In one embodiment the cells are autologous and the TCRs are allogeneic. In some embodiments, the TCRs are autologous and the T cells are allogeneic. In some embodiments, the TCRs are autologous and the T cells are autologous. Preferably, the cells are autologous to the subject.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount or any level of treatment or prevention of cancer in a mammal.

Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

With respect to the inventive methods, the cancer can be any cancer, including any of sarcomas (e.g., synovial sarcoma, osteogenic sarcoma, leiomyosarcoma uteri, and alveolar rhabdomyosarcoma), lymphomas (e.g., Hodgkin lymphoma and non-Hodgkin lymphoma), hepatocellular carcinoma, glioma, head-neck cancer, acute lymphocytic cancer, acute myeloid leukemia, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer (e.g., colon carcinoma), esophageal cancer, cervical cancer, gastrointestinal cancer (e.g., gastrointestinal carcinoid tumor), hypopharynx cancer, larynx cancer, liver cancer, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, rectal cancer, renal cancer, small intestine cancer, soft tissue cancer, stomach cancer, testicular cancer, thyroid cancer, ureter cancer, and urinary bladder cancer. In some embodiments, the cancer is a solid tumor.

The method may comprise combining the cell population of tumor-reactive T cells expressing subject specific TCRs with a pharmaceutically acceptable carrier to obtain a pharmaceutical composition comprising a personalized cell population of tumor-reactive T cells. Preferably, the carrier is a pharmaceutically acceptable carrier. With respect to pharmaceutical compositions, the carrier can be any of those conventionally used for the administration of cells. Such pharmaceutically acceptable carriers are well-known to those skilled in the art and are readily available to the public. It is preferred that the pharmaceutically acceptable carrier be one which has no detrimental side effects or toxicity under the conditions of use. A suitable pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an embodiment, the pharmaceutically acceptable carrier is supplemented with human serum albumen.

The T cells can be administered by any suitable route as known in the art. Preferably, the T cells are administered as an intra-arterial or intravenous infusion, which preferably lasts approximately 30-60 min. Other examples of routes of administration include intraperitoneal, intrathecal and intralymphatic. T cells may also be administered by injection. T cells may be introduced at the site of the tumor.

For purposes of the invention, the dose, e.g., number of ceils in the inventive cell population expressing subject specific TCRs, administered should be sufficient to effect, e.g., a therapeutic or prophylactic response, in the subject over a reasonable time frame. For example, the number of cells should be sufficient to bind to a cancer antigen, or detect, treat or prevent cancer in a period of from about 2 hours or longer, e.g., 12 to 24 or more hours, from the time of administration. In certain embodiments, the time period could be even longer. The number of cells will be determined by, e.g., the efficacy of the particular ceils and the condition of the subject (e.g., human), as well as the body weight of the subject (e.g., human) to be treated.

Typically, the attending physician will decide the number of the cells with which to treat each individual patient, taking into consideration a variety of factors, such as age, body weight, general health, diet, sex, route of administration, and the severity of the condition being treated. By way of example and not intending to limit the invention, the number of cells can be about 10×10⁶ to about 10×10¹⁰ cells per infusion, about 10×10⁹ cells to about 10×10¹⁰ cells per infusion. The ceil populations obtained by the disclosed methods may, advantageously, make it possible to effectively treat or prevent cancer. Likewise, any suitable dose of T cells can be administered.

As an alternative, or in addition to, adoptive therapies, contemplated herein are non-adoptive methods of treatment involving modified T cells in accordance with the disclosure. In some embodiments, these methods comprise administering an inhibitor (e.g., a transient inhibitor) of TCF7 expression to a patient, such as through systemic administration. The objective of such methods is to provide upregulation of TCF7 in the T cells of the subject in vivo.

### Examples

The function and advantage of these and other embodiments of the present disclosure will be more fully understood from the Examples below. The following Examples are intended to illustrate the benefits of the present disclosure and to describe particular embodiments, but are not intended to exemplify the full scope of the disclosure. Accordingly, it will be understood that the Examples are not meant to limit the scope of the disclosure.

### Example 1: Development of Methods to Expand Antitumor T Cells with Stem-Cell Memory Phenotype

Many protocols are capable of expanding tumor specific T cells in vitro, usually resulting in a large number of effector T cells that can be adoptively transferred to kill tumor cells. However, the ability of T cells to persist over time and continue killing tumor cells depends greatly on their phenotypic and functional state. Several studies have shown that Tscm (stem-cell like T cells), defined largely by expression and function of the TCF7 transcription factor, are much more persistent and as a result more effective in vaccine-mediated protection (Baharom et al. 2021) and anti-PD-1-induced tumor clearance (Siddiqui et al. 2019; Lin et al. 2016; Kurtulus et al. 2019) (FIG. 1B). Recent studies found that adoptive T cell infusion products are most effective when they contain a high frequency of Tscm (Krishna et al. 2020). It was found that patients receiving immunotherapy achieved better control of melanoma in the presence of more TCF7+ T cells in human and mouse tumors (Sade-Feldman et al. 2018) (FIG. 1A). To identify the genes that regulate T cell TCF7 expression following in vitro expansion, unbiased whole genome CRISPR screens were applied and TCF7 regulators were identified. Presented herein is a study of the identified regulators of TCF7 from genome-wide screens and their impact on Tscm phenotype and function both in vitro and in vivo and the application of those findings to optimize the preparation of T cells expressing tumor-reactive TCRs for adoptive transfer.

Tcf7DTR-GFP P14 chimeric mice harboring B16-gp33 tumors of around 100 mm³ were exposed to FTY720 (FTY) (to prevent immigration of new lymphocytes into the tumor). Tcf7GFP-DTR+P14 cells were ablated using DT and treatment with anti-PD1/CTLA4 Abs checkpoint blockade immunotherapy (CIB) or isotype control Ab was initiated (see FIGS. 1A and 1B). This data indicates that TCF7-expressing (TCF7+) cells correlate with response to anti-PD1 in melanoma, and mouse MC38 tumor response to checkpoint blockade immunotherapy (CBI) varies proportionately with TCF7 expression in cells.

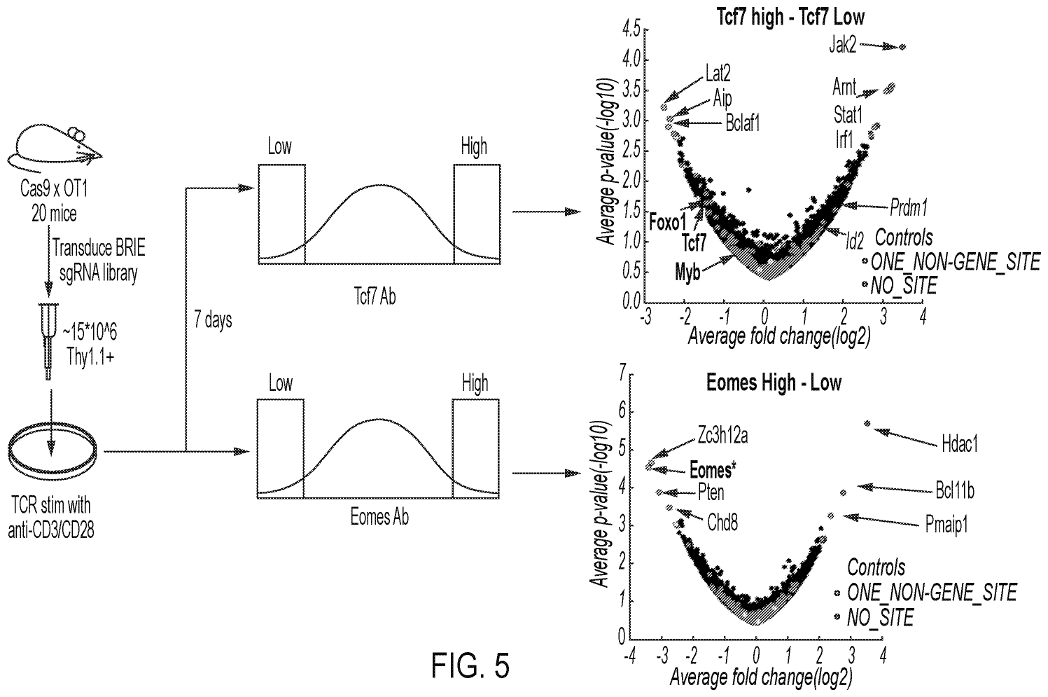

Several genome-wide CRISPR screens were performed to identify genes that regulate TCF7 expression and identified JAK2 and STAT1 as negative regulators (FIG. 5). Additional experiments demonstrated that deletion of these genes does not affect T cell activation or proliferation, leading to the hypothesis that these genes are candidate targets for inhibition to increase the number of TCF7+ cells in expanded infusion products, and thus maximize the efficacy of the product in vivo.

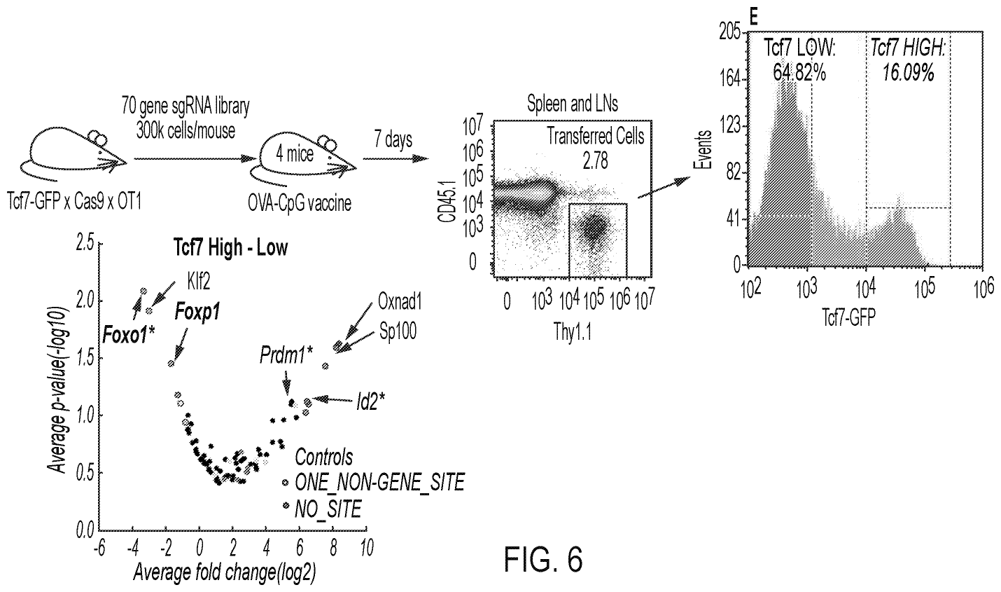

The FACS results of the genome-wide in vitro screens shown in FIG. 5 indicated that JAK2 and STAT1 were strong negative regulators of TCF7. Furthermore, the FACS results of the genomic in vitro screens shown in FIG. 6 implicated novel in vivo-specific regulators of TCF7, such as Klf2, Oxnad1, and Sp100.

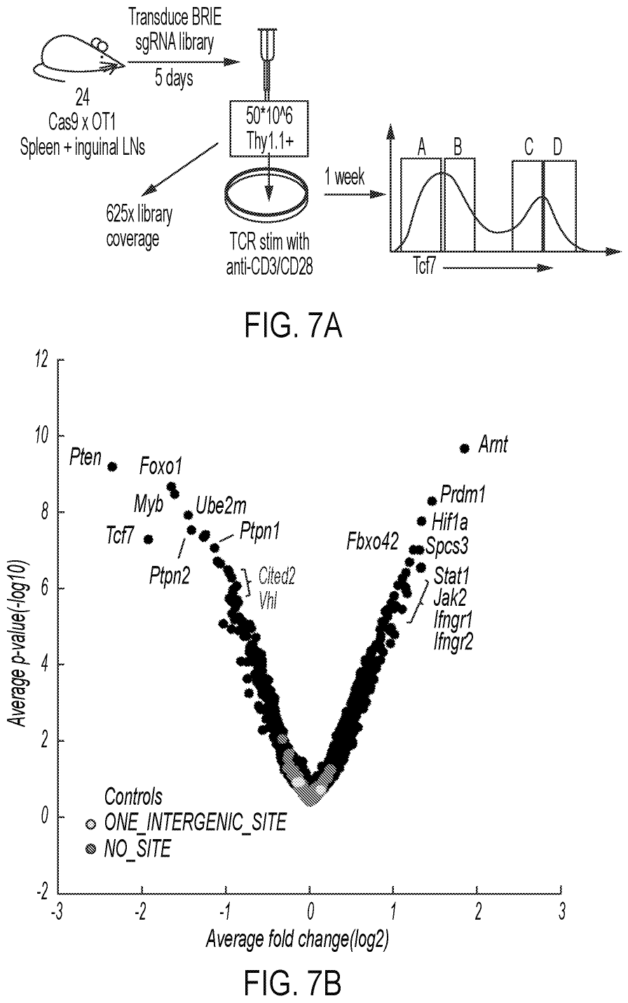

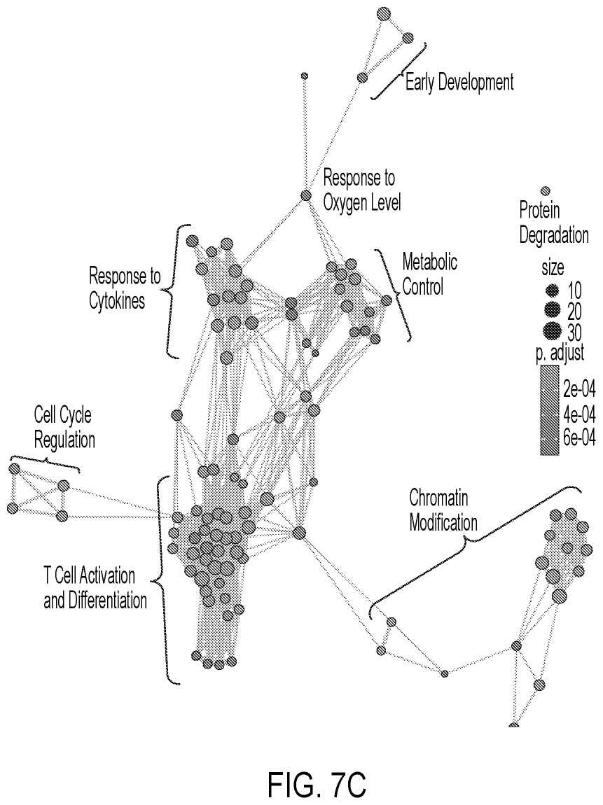

Naïve CD8 T cells were isolated from Cas9/OT1 double transgenic mice, then transduced with a whole genome sgRNA lentiviral library modified to have both VSV-g and ecotropic (MLV) envelope proteins. The lentiviral library used was a mouse CRISPR BRIE pooled library (see FIG. 7A). Five days after transduction, Thy1.1+ transduced cells were purified using a two step staining and magnetic column separation. The purified transduced cells were then activated in vitro using plate-bound anti-CD3 and anti-CD28, expanded for 1 week, then sorted by Tcf7 expression using intracellular Tcf7 antibody staining. Genomic DNA was isolated from each sorted population to allow amplification of sgRNA sequences and quantification of sgRNA abundance in Tcf7 high and Tcf7 low cells. The results of this screen are shown in FIGS. 7A-7C.

### Enhanced Tscm Phenotype and Anti-Tumor Efficacy by Upregulating TCF7.

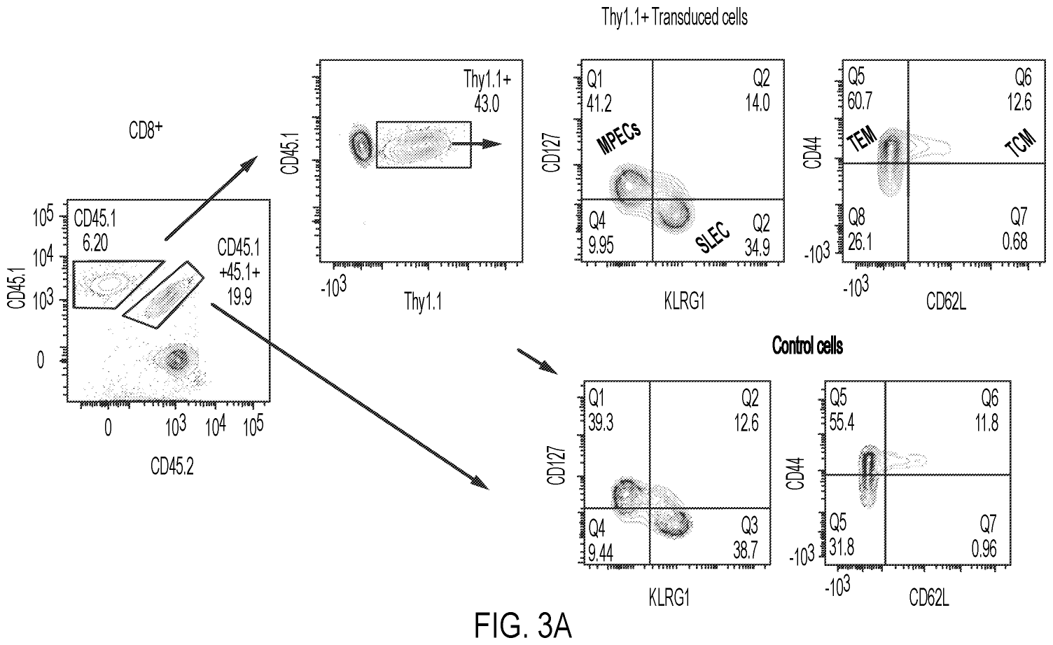

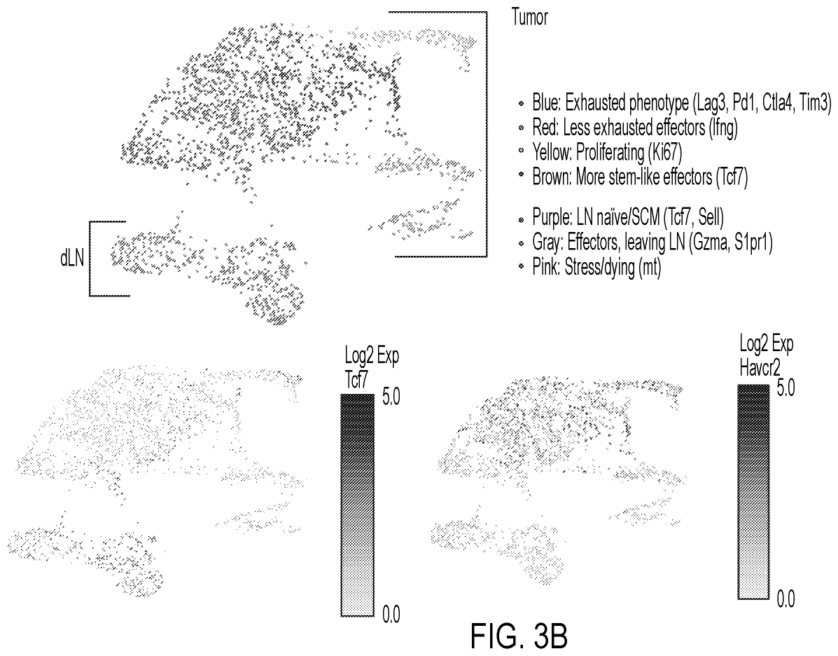

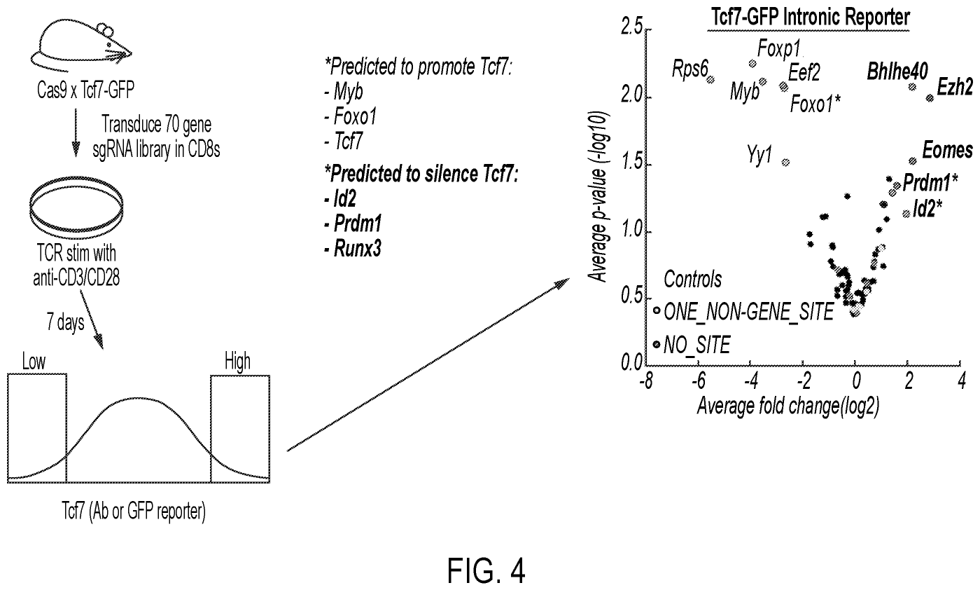

First, a targeted pooled screen was performed to determine how each of the TCF7 regulators from the initial screen (e.g., top 40 hits) impacted long-term persistence and proliferative potential of T cells in mice. A reversible perturbation method (doxycycline-inducible sgRNAs co-expressed with dCas9-KRAB) was developed and used for transient reduction of inhibitors of Tscm while avoiding impact on later T cell activation when the T cell encounters tumor. OVA-specific OT1 T cells with transiently reduced negative regulators of TCF7 were transferred into mice harboring OVA+ tumors, and sgRNA abundance was measured in cytolytic T cells in tumors and in lymph nodes at several time points (FIG. 3B). Top enriched sgRNAs were then individually tested for their ability to enable a large proliferative burst in response to anti-PD1 treatment, control tumor growth, keep mice alive, and protect against later tumor rechallenge.

The map of expression in the tumor (FIG. 3B) shows that the disclosed methods identify transduced, antigen specific T cells in both the tumor and draining lymph node that recapitulate T cell states observed in other studies, notably forming both Tcf7+(high TCF7 expression) and Tcf7-(low TCF7 expression) subsets in the tumor and draining lymph node as identified by hashing antibodies. The T cells developed into the expected subtypes.

Mice were implanted with MC38 tumors in flank, T cells transduced and 5 days after transduction (1 week after tumor implant) transferred to tumor bearing mice. Tumors and tissue-draining lymph nodes (dLNs) were harvested 1 week later, CD8+ T cells were isolated by magnetic activated cell sorting (MACS) and then sorted on Thy1.1-PE (FIG. 3A).

Adoptive Transfer of Human Tscm Cells Induced with Modulators of TCF7.

Inhibitors of the top TCF7 negative regulators, including JAK2, are tested using a human adoptive T cell transfer system developed previously. However, transient inhibitors of negative regulators of TCF7 are added during in vitro expansion (e.g., using siRNAs, dCas9/RNP or small molecules such as FDA-approved JAK2 inhibitors). Expanded T cells are transferred into mice and their ability to clear xenografts and persist are assessed.

Naïve T Cell Transduction with Ecotropic (ECOG) Pseudotyped Lentivirus

In an exemplary lentiviral transduction method, P14 T cells were transferred to mice infected with acute lymphocytic choriomeningitis virus (LCMV), Armstrong strain. T cells were transduced, 5 days later transferred to recipient mice that were then infected with LCMV. Cells were analyzed at 7 and 30 days. The results shown in FIG. 3A indicate that transduced T cells expand and contract in line with co-transferred fresh naïve cells, maintaining the same cell number ratio and adopting the same effector and memory phenotypes.

In some aspects, provided herein are lentiviral particles comprising a recombinant lentiviral vector comprising one or more polynucleotides encoding a) a CRISPR-associated nuclease, and b) a plurality of guide RNA molecules, wherein each of the guide RNA molecules comprises a different guide sequence of at least 10 contiguous nucleotides that is complementary to a target sequence in the genomic DNA of a T cell, and wherein the lentiviral particle comprises VSV-g and ecotropic (ECO) envelope proteins.

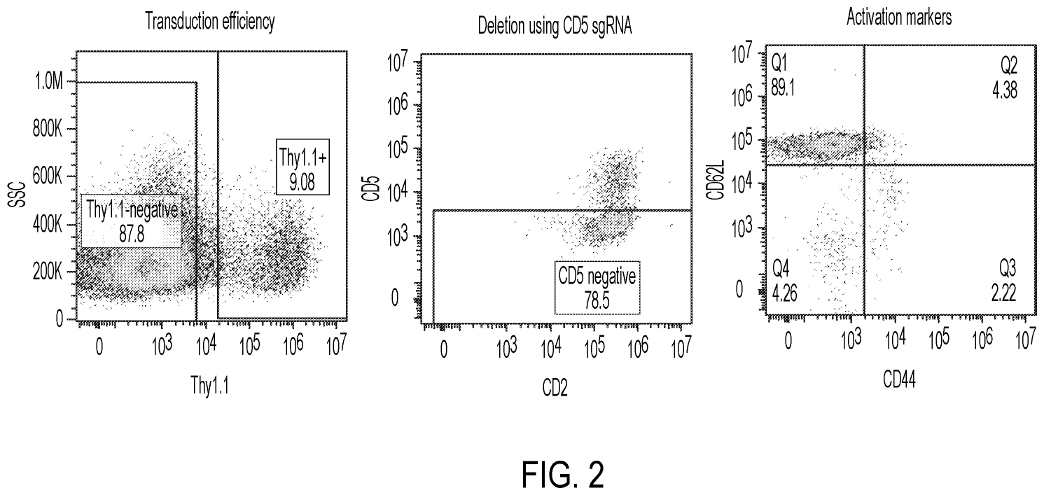

To transduce naïve CD8+ T cells without the need for activation or stimulation (see FIG. 2) the following exemplary procedure is used:

### Step 1: Virus Production,

Reagents:
Dul′ecco's Modification of 'agle's Medium (VWRL0111-0500)
Fetal Bovine Serum (Sigma Cat. #F4135)
Microbiology-grade Bovine Serum Albumin (VWR Cat. #14230-738)
Penicillin Streptomycin (VWR Cat. #97063-708)
293x cells (Takara 632180)
PBS −/− (Thermo Fisher Scientific 10010049)
Trypsin-EDTA (Fisher Scientific 25-300-054)
Opti-MEM (Thermo Fisher Scientific 31985062)
pDNA (sgRNA containing lentiviral vector)
pPAX2 (Addgene 12260)
pVSVG ( ) (Addgene 12259, VSV-G envelope protein)
pCAG-Eco (Addgene 35617, ecotropic, MLV envelope protein)
LT-1 (Mirus Bio MIR2305)

Part 1: Seeding Cells for Transfection

Seed 293x cells (Monday 1 PM):
1. Work in small batches (3-4 T175s) so that cells do not get over-trypsinized.
2. Aspirate media, then gently wash with 10 mL PBS −/− per T175 and aspirate PBS. If the cells are starting to lift off without trypsinization, the PBS wash will appear cloudy and should not be used for virus production.
3. Add 6 mL trypsin per T175 and rock to cover the surface. Allow to trypsinize in the hood for less than 2 min.
4. Quench trypsin with 6 mL of media per T175 and collect cell suspension into a conical.
5. Wash each T175 with 6 mL media and collect wash in the same conical.
6. Thoroughly mix cell suspension and count with trypan blue.
7. Seed 18 million cells in 30 mL media per T175+2 more T175s than needed, i.e., 22 T175s for a 20-flask transfection.

Part 2: Transfection

A: Prepare reagents (Tuesday AM):
1. Check a T175 for confluency, they should be around 60%.
2. Warm Opti-MEM in water bath, take LT-1 out of 4° C. and allow to come to room temp and thaw out packaging plasmids and plasmid DNA.
3. Calculate the amounts of plasmids needed for the transfection+1 T175 extra per table shown below:

| Reagent | Amount needed per T175 | |
|---|---|---|
| pDNA | 40 | μg |
| pPAX2 | 50 | μg |
| pVSVG envelope | 2.5 | μg |
| pCAG-eco | 2.5 | μg |
| OptiMEM | 50 | μL |

4. Make a master mix of all three common reagents. Add pDNA separately.
5. Divide the master mix total volume by the number of transfection T175s to calculate the volume per flask. Aliquot this volume into sterile microcentrifuge tubes, one for each T175.
6. Aliquot 6 mL of Opti-MEM into a 15 mL conical, one for each T175.

B: Perform transfection(s) (Tuesday AM):
1. Gently pipette 305 μL LT-1 (1 μg/μL) into the first Opti-MEM conical. Carefully invert the conical 5-7 times, avoiding bubbles. Let the mixture sit for 3 minutes.
2. Gently pipette the plasmid mix into the LT-1+Opti-MEM conical. Carefully invert the tube 5-7 times, avoiding bubbles. Let sit at room temperature for 27-30 minutes.
3. Subsequent transfection conicals can be processed with 3-min intervals to optimize time.
4. Change the pipette-aid to slow release.
5. After 27-30 min, gently take out a T175 from the incubator.
6. Slowly drip the plasmid+LT-1+Opti-MEM mixture in a zig-zag or circular motion over the surface of the cells. Make sure to cover as much surface area as possible.
7. Carefully place the T175 back into the incubator without moving the media too much.
8. Note time of transfection completion in order to change the media after 6-8 hrs.

C: Change media (Tuesday PM):
1. 6-8 hours post-transfection, carefully aspirate the media.
2. Gently replace with viral harvest media (DMEM+10% FBS+1% BSA+1% Pen/Strep) by pipetting down the top side (not on the cells) of the T175 with a slow dispense speed. Use 60 mL of harvest media per T175 for a high-titer vector and 40 mL of harvest media per T175 for low-titer vectors.
3. Gently move the T175 back to the incubator. Note the time of the media change.

Part 3: Harvesting and Aliquoting Virus

A: Collect virus 36 hours post-transfection (Thursday AM):
1. Choose appropriately sized aliquots to avoid freeze/thawing the virus and label the tubes.
2. Carefully pipette viral supernatant from each T175 into a 50 mL conical, splitting across multiple conicals if necessary.
3. Spin at 230 g for 1 min to pellet 293x cell debris.
4. Avoiding the cell pellet, pipette off viral supernatant into a new 50 mL conical, or a 500 mL or 1 L sterile bottle. Only fill bottle(s) up to 75%. Note do not filter the virus.
5. If multiple bottles are needed, mix the virus by pouring from one bottle to the next, until homogeneous.
6. Aliquot virus into labeled tubes.
7. Freeze at −80° C.

### Step 2: Virus Titer

Titer on T cells, mouse cancer cell line (MC38), or 293x depending on goal. To titer on MC38/293x plate cells with titrated virus and polybrene 10 μg/mL. Readout transduction efficiency using flow cytometry 2-3 days later. To titer on T cells follow steps below.

### Step 3: Virus Plating

Reagents:
Non-TC coated flat-bottom multiwell plates (6, 24, or 96 wells depending on experimental goals)
   6 well: VWR 15705-056
   24 well: Corning 351147
   96 well: VWR 15705-066
RetroNectin® (Takara Bio T100B)
PBS −/− (Thermo Fisher Scientific 10010049)
Microbiology-grade Bovine Serum Albumin (VWR Cat. #14230-738)

Plating
1. Coat non TC coated plate with PBS −/− diluted RetroNectin (0.1 mg/mL) overnight. Note this is frozen in aliquots in diluted form. Recommended range is 4-20 μg retronectin/cm².
   6 well: Surface area 9.5 cm². Plate 1 mL well->0.1 mg/well->˜10 μg/cm².
   24 well: Surface area 2 cm². Plate 300 μL well->0.03 mg/well->˜15 μg/cm².
   96 well: Surface area 0.32 cm². Plate 50 μL well->0.005 mg/well->˜16 μg/cm².
2. Allow to incubate at 4° C. overnight.
3. Remove retronectin and block retronectin plate with 2% BSA in PBS −/− for 30 min at RT.
   6 well: 2 mL blocking volume
   24 well: 0.5 mL blocking volume
   96 well: 100 μL blocking volume
4. While the plate is blocking, remove the virus from the freezer and thaw it at 37° C., removing from heat source as soon as or just before it is finished thawing.
5. Remove some of the BSA buffer such that once the virus is added there will be the same final volume in the plate. Add the virus to the plate. Final volumes should be:
   6 well: 2 mL final volume
   24 well: 0.5 mL final volume
96 well: 100 μL final volume
6. Centrifuge plate for 2 hours at 32° C. at 2000 g, Accel-9, Brake-4. If spin is finished before cells are ready, leave the plate in the hood at RT with liquid in the wells so the wells do not dry out.

Step 4: Naïve CD8+ T Cell Isolation

Reagents:
RPMI 1640 with L-Glutamine (Fisher Scientific 11-875-093)
Fetal Bovine Serum (Sigma Cat. #F4135)□
Penicillin Streptomycin (VWR Cat. #45000-652)
Cas9×P14/OT1 Donor mice
PBS −/− (Thermo Fisher Scientific 10010049)
EDTA 0.5 M (VWR 45001-122)
Microbiology-grade Bovine Serum Albumin (VWR Cat. #14230-738)
Naïve CD8 T cell isolation kit (Miltenyi Biotec 130-096-543)
LS columns (Miltenyi Biotec 130-042-401)

Preparation

While the virus is spinning; keep Miltenyi beads on ice while not using, vortex or mix well before adding beads to cells.
1. Sacrifice animals and isolate spleens
2. Dissociate spleen with frosted slides then filter through a 70 micron filter and wash with RPMI 1% FBS buffer. Spin 300×G 5 min 4° C. Accel-9, Brake-9.
3. Remove supernatant and resuspend splenocytes in 400 μL MACS buffer (PBS −/−, 2 mM EDTA, 0.5% BSA).
4. Add 100 μL of Naïve CD8 antibody cocktail to cells+mix well+incubate at 4° C. for 5 minutes (this contains biotinylated antibody mixture to other immune lineages other than CD8).
5. After incubation, add 200 μL MACS, followed by 200 μL of anti-biotin+100 μL anti-CD44. Incubate for 10 min at 4° C.
6. Add at least 10 mL of MACS to wash and spin down at 300×G 5 min 4° C. Accel-9, Brake-9.
7. While spinning, rinse MACS columns with 3 mL of MACS buffer.
8. Prepare collection tubes (15 mL conical).
9. Resuspend cell pellet in 0.5 mL of MACS, pass through filter/column.
10. Rinse tube with 3 mL of MACS+add that to the column.
11. Once all the cells drip through, add 5 mL MACS to 15 mL conical and spin down at 300×G 5 min 4° C. Accel-9, Brake-9.
12. Resuspend cell pellets in complete media (RPMI+10% FBS+1% PS+10 mM HEPES+55 uM BME) and count live cells with trypan blue. Per spleen should get 5-20 million cells (depending on P14 vs OT1.

### Step 5: Naïve CD8+ T Cell Transduction

Reagents:
RPMI 1640 with L-Glutamine (Fisher Scientific 11-875-093)
Fetal Bovine Serum (Sigma Cat. #F4135)□
Penicillin Streptomycin (VWR Cat. #97063-708)
HEPES 1M (VWR 97064-360)
BME (VWR 97064-588)
Recombinant Murine IL7 (Peprotech 217-17-10 μg)
Recombinant Murine IL15 (Peprotech 210-15 μg)
LentiBOOST P® (Sirion Biotech)

Transduction
1. Add naïve T cells to the plates in complete R10 media (RPMI+10% FBS+1% PS+10 mM HEPES+55 uM BME) containing IL7/IL15 at 10 ng/mL each and lentiboost (Stock is 100 mg/mL and is recommended at 1:100, final concentration 1 mg/mL).
   6 well: 1-4 million cells in 2 mL of complete media with cytokines/lentiboost
   24 well: 250K cells in 300 μL of complete media with cytokines/lentiboost
   96 well: 50K cells in 100 μL of complete media with cytokines/lentiboost

### Step 6: Culture

Reagents:
RPMI 1640 with L-Glutamine (Fisher Scientific 11-875-093)
Fetal Bovine Serum (Sigma Cat. #F4135)
Penicillin Streptomycin (VWR Cat. #97063-708)
HEPES 1M (VWR 97064-360)
BME (VWR 97064-588)
Recombinant Murine IL-7 (Peprotech 217-17-10 μg)
Recombinant Murine IL-15 (Peprotech 210-15 μg)

Culture
1. Place in a 37° C. incubator.
2. 48 hours after plating T cells on retronectin/lentivirus, change the media to get out of lentiboost and off the retronectin plates. Use extra complete media to wash the well and dilute the lentiboost. Spin down at 4° C. for 5 min at 300×G, Accel-9, Brake-9 and replate in fresh complete media with IL-7 and IL-15 at 10 ng/mL. When replating the cells should be at a density of 1-2 million cells/mL. Here depending on the number of cells TC flasks can be used.
3. Move to a new plate/flask and culture in complete media for an additional 3 days with IL-7/IL-15 at 10 ng/mL each.
4. Readout transduction percentage by flow cytometry 5 days post transduction.

### Example 2: Effects of Inhibition of IFN-Gamma Receptor on TCF7 Expression and T Cell Persistence In Vivo

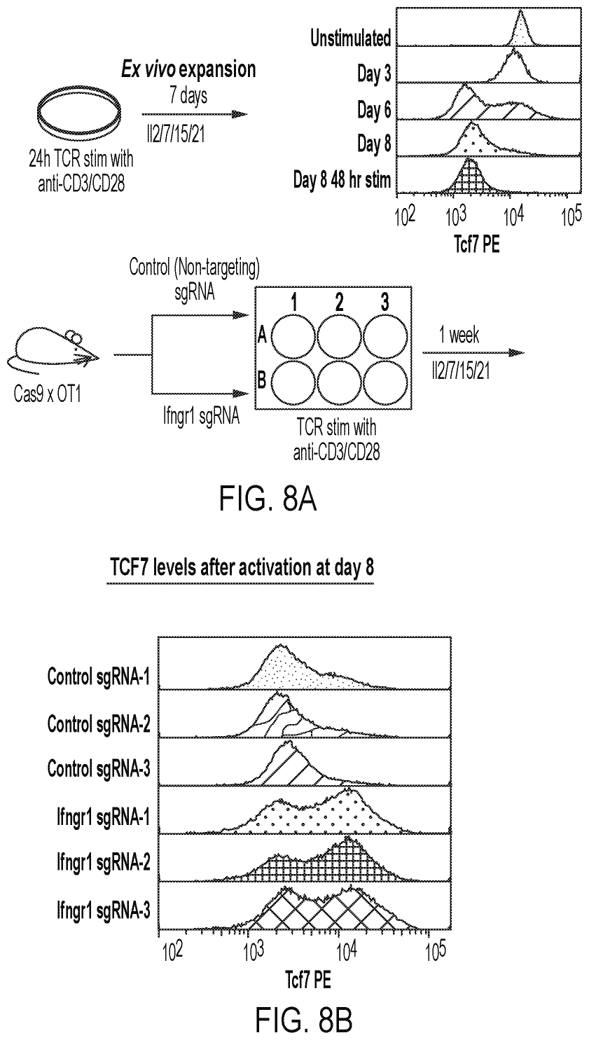

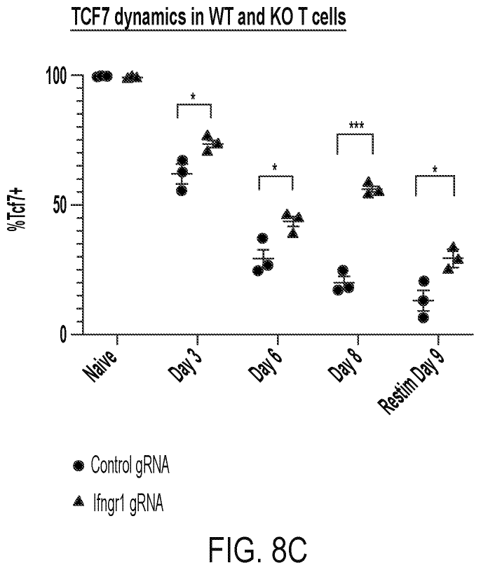

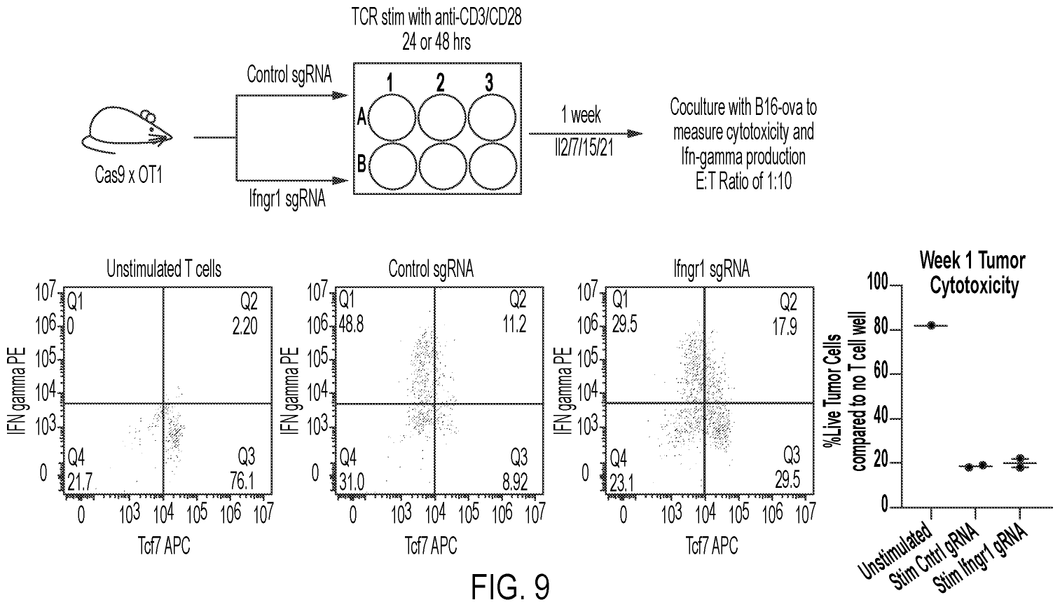

Naïve CD8 cells were isolated from mice, after which they were stimulated with anti-CD3 and anti-CD28 antibodies that were bound to flat-bottom plates for 24 hours, and then cultured in the presence of recombinant IL-2, IL-7, IL-15, and IL-21. Tcf7 protein levels were monitored using intracellular staining with anti-Tcf7 antibodies and analyzed by flow cytometry. To validate one of the newly identified Tcf7 regulators, naïve CD8 cells were isolated from Cas9/OT1-double transgenic mice then transduced with lentiviral vectors containing either control (non-targeting) sgRNA or an Ifngr1-targeting sgRNA. As shown in FIG. 8A, after purification of Thy1.1+ transduced cells five days after transduction, cells were stimulated with anti-CD3/anti-CD28 plate-bound antibodies and cultured in IL2, IL7, IL15, and IL21 for 1 week. As shown in FIGS. 8B and 9C, knock-out of the Ifngr1 gene leads to retention of Tcf7 in a majority of CD8+ cells 8 days after initial stimulation and restimulation. One cohort that was restimulated with anti-CD3/anti-CD28 one week after the initial stimultation, and then analyzed 9 days after restimulation. FIG. 8C shows the quantification of this data at various time points after stimulation.

OT1 T cell intracellular staining of Tcf7 and IFN-gamma was measured two days after a co-culture with B16-OVA melanoma cells to measure the levels of tumor cytotoxicity and IFN-gamma production. The B16-OVA cell line expresses ovalbumin (OVA) in order to facilitate strong immune responses to tumor antigens. As shown in FIG. 9, prior to co-culture, T cells received either control (non-targeting) or IFN-γr1 (Ifngr1)-targeting sgRNAs and were activated by anti-CD3/anti-CD28 stimulation 1 week prior to co-culture, with the exception of the non-stimulated group which received no anti-CD3/CD28 stimulation. Flow cytometry plots of TCF7-presenting cells (APCs) are also shown in FIG. 9.

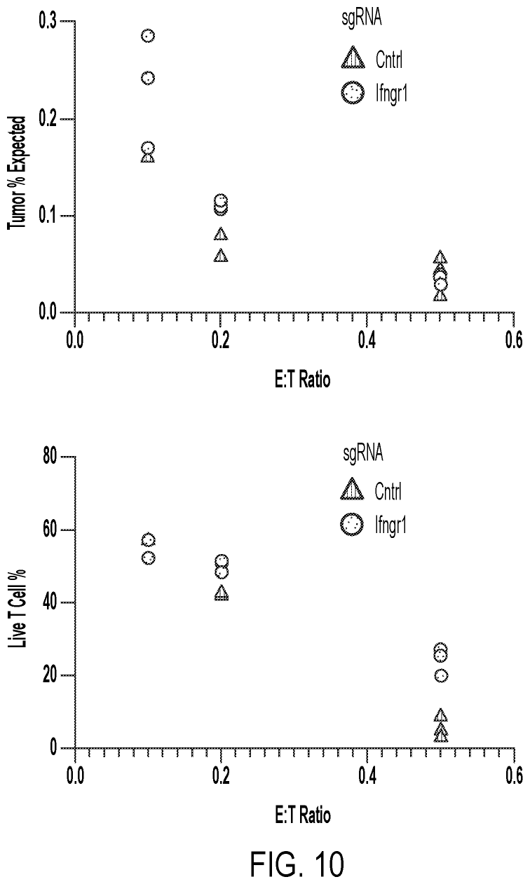

Naïve CD8 T cells that received either control or Ifngr1-targeting sgRNAs were co-cultured with MC38-OVA tumor cells, and two days later the number of live tumor cells and the viability of T cells was quantified, as shown in FIG. 10. Like B16-OVA, the MC38-OVA line expresses ovalbumin. Experimental wells contained T cells at effector:target (E:T) ratios of 1:10, 1:5, and 1:2. Ifngr1-deficient CD8s were able to kill tumor cells at all tested E:T ratios, although tumor killing was somewhat less efficient at lower E:T ratios. As shown in the right panel of FIG. 10, at higher E:T ratios, a superior survival of Ifngr1-deficient T cells is observed.

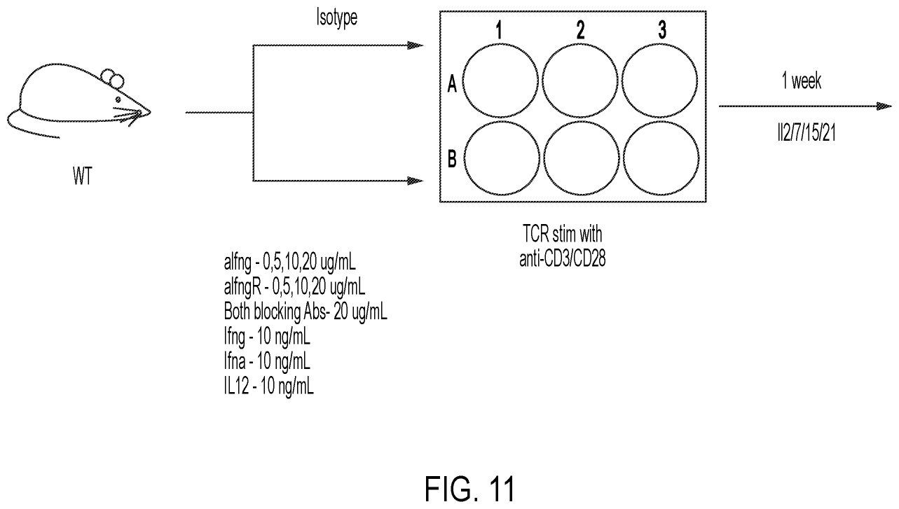

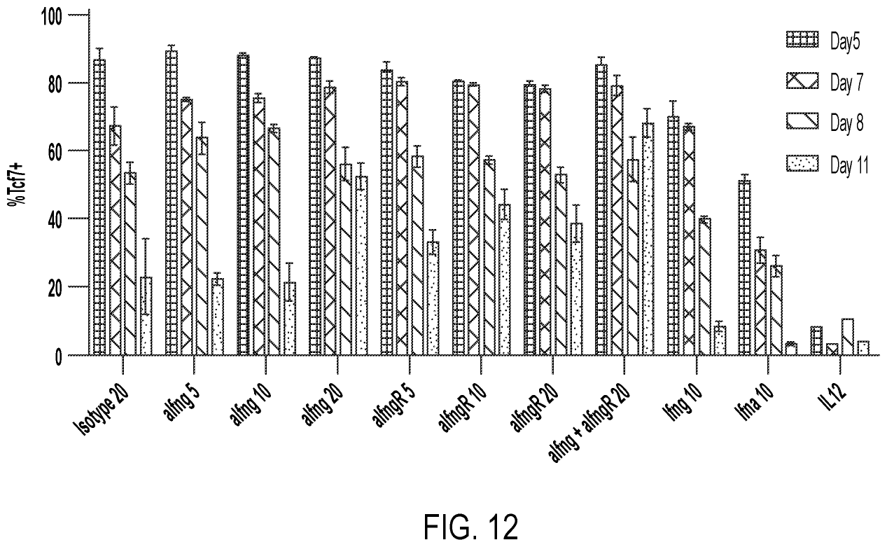

Next, it was sought to determine whether the effects of blocking IFN-γ signaling on the Tcf7+ phenotype could be recapitulated using a method separate from CRISPR-deletion, such as with neutralizing antibodies. As such, antibodies to both the Ifn-gamma and Ifn-gamma receptor genes were used. Naïve CD8 T cells were isolated from wild-type mice and cultured in the presence of either isotype-control or a combination of Ifn-gamma and IfngR neutralizing antibodies at various dosing levels or in the presence of added cytokines (either IFN-gamma, IFN-alpha, or IL12), then stimulated with anti-CD3/anti-CD28 antibodies and cultured for 1 week in IL2, IL7, IL15, and IL21, and Tcf7 protein levels were quantified by intracellular flow cytometry, as shown in FIG. 11. Addition of interferon-γ to the T cells had no effect on Tcf7 expression. The data shown in FIG. 12 demonstrates that the combination of anti-Ifng and anti-IfngR neutralizing antibodies effectively prevents most of the Tcf7 silencing present when isotype (control) antibodies were used.

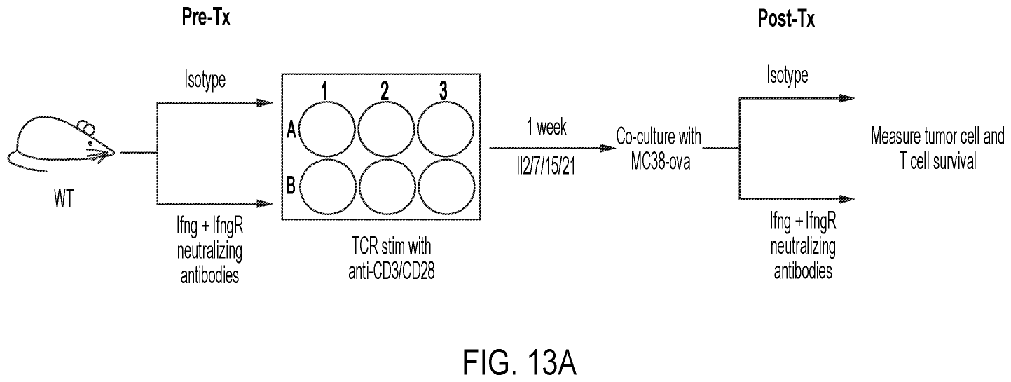

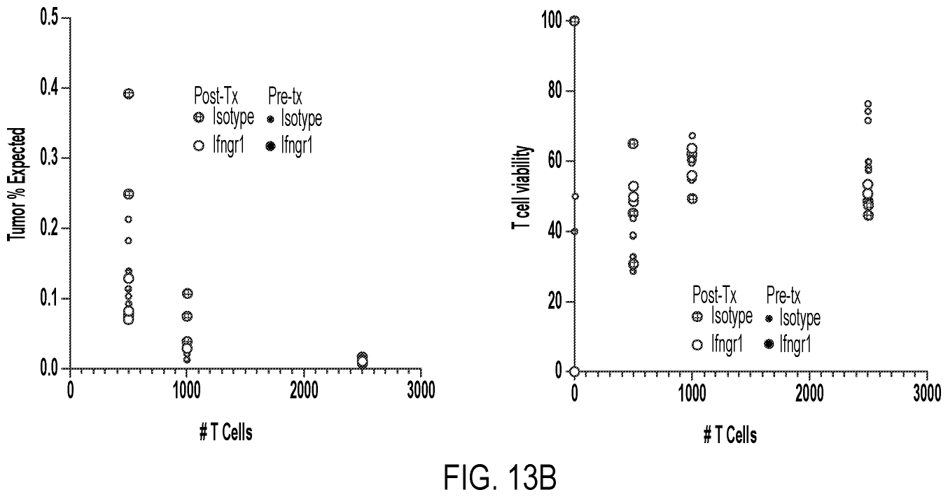

Next, CD8 T cells were isolated from WT mice and treated with isotype or a combination of anti-Ifng and anti-IfngR neutralizing antibodies. Cells were stimulated with anti-CD3/anti-CD28 antibodies and cultured for 1 week in a mixture of recombinant interleukins: IL-2, IL-7, IL-15, and IL-21. Cells were then co-cultured with MC38-OVA tumor cells at various E:T ratios, and tumor cell cytotoxicity was subsequently measured. Antibody treatment was varied both before tumor co-culture (pre-transfer (“Pre-Tx”)) and during the tumor co-culture (post-transfer (“Post-Tx”)), thus there were four treatment groups: 1) Isotype antibodies both pre and post tumor co-culture, 2) anti-Ifng/anti-IfngR antibodies both pre and post tumor co-culture, 3) Isotype antibody pre- and neutralizing antibodies post-tumor co-culture, 4) Neutralizing antibodies pre- and isotype antibody post tumor co-culture. As indicated by the live tumor cell data shown in FIG. 13B, this data demonstrates that CD8 T cells retain cytotoxicity in the presence of anti-Ifng/anti-IfngR antibodies. This data further shows that T cell survival is improved when anti-Ifng and/or anti-IfngR antibodies are present during tumor co-culture. Best T cell survival was observed following treatment with isotype antibody pre-transfer and anti-IfngR1 antibody post-transfer.

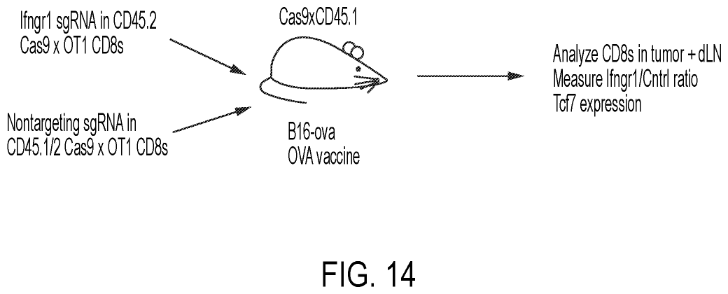

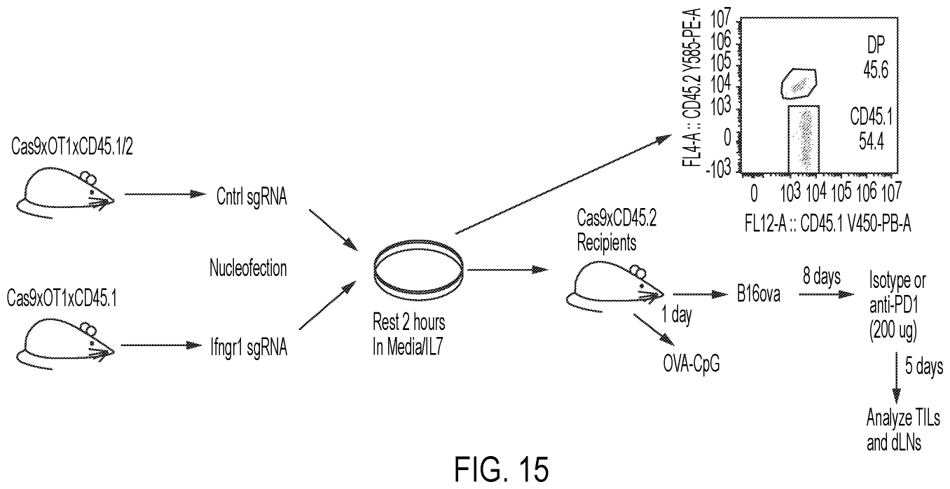

Subsequently, an experiment was performed to determine whether deletion of Ifngr1 provides a competitive advantage for CD8 T cells in vivo in the setting of either vaccination or tumors, as shown in the experimental schematics shown in FIGS. 14 and 15. Congenically marked CD8 T cells are isolated from Cas9/OT1-double transgenic mice and either control or Ifngr1-targeting sgRNAs are delivered by nucleofection as described in Majumder, et al. Front. Immunol., 2021. Cells from both conditions are then combined and transferred to new recipient mice. Using the congenic CD45.1 and CD45.2 surface markers, cells with non-targeting sgRNA, Ifngr1 sgRNA, and recipient mouse T cells can be distinguished by flow cytometry. Recipient mice are either vaccinated with an OVA-CpG immunization or have B16-OVA tumors implanted. Mice with tumors were treated with either isotype or anti-PD1 antibodies 8 days after tumor implant.

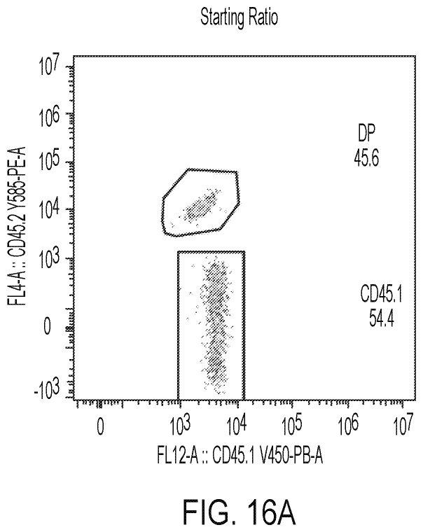

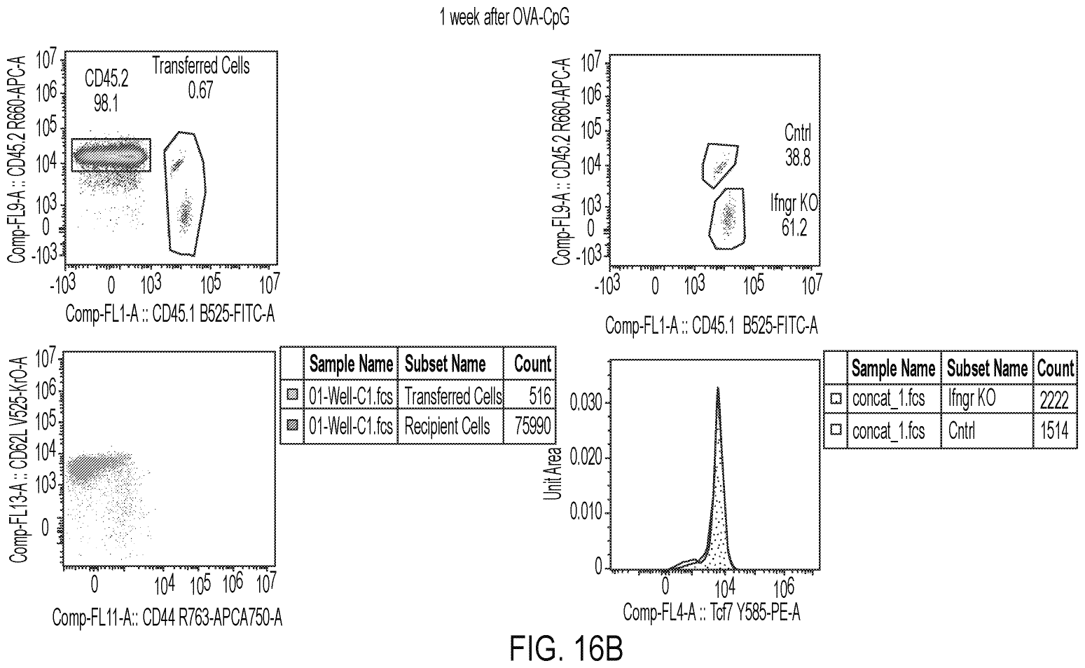

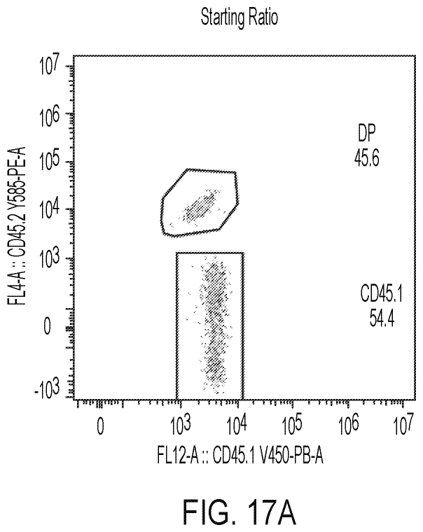

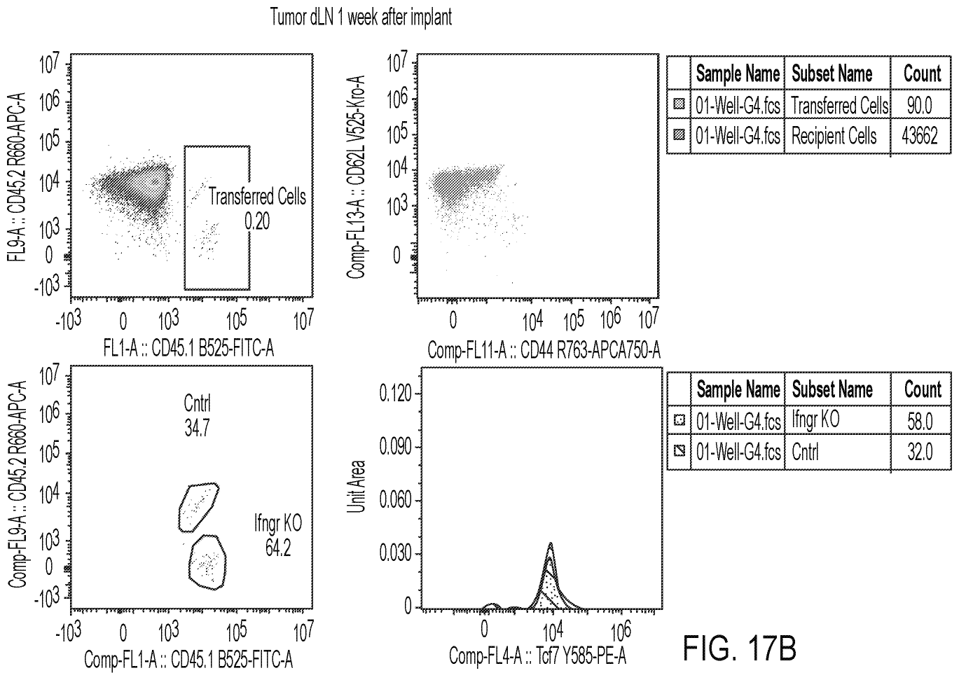

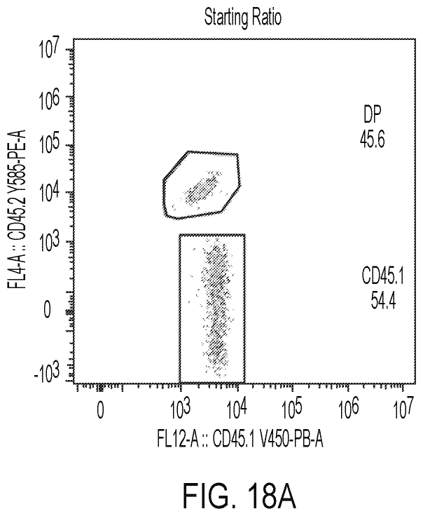

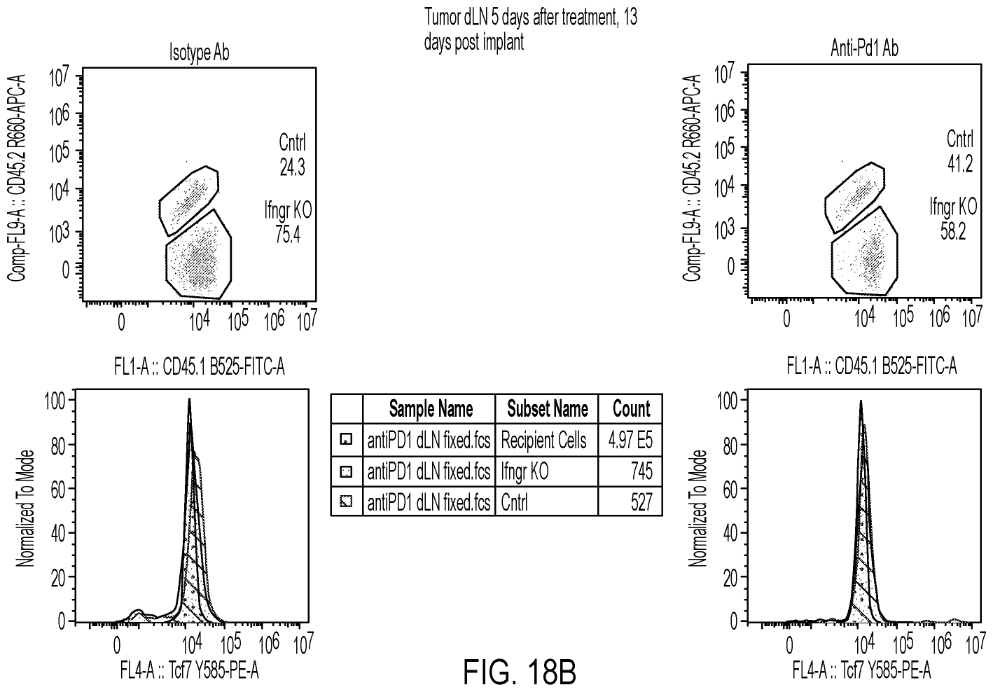

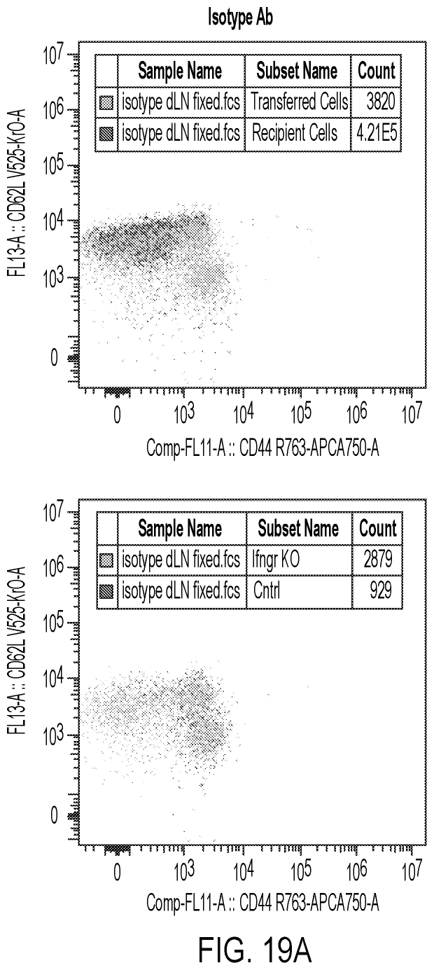

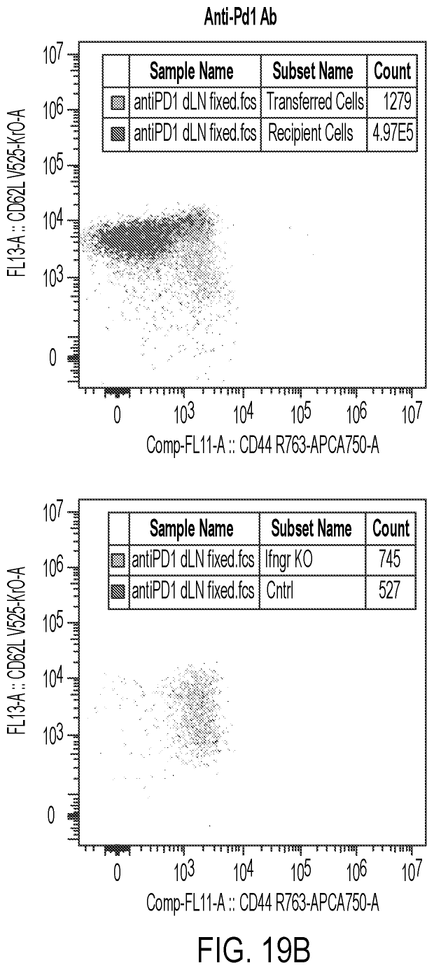

Subsequently, the competitive advantages for Ifngr1-sgRNA cells following transfer of CD8+ T cells containing control or Ifngr1-sgRNA containing CD8 T cells one week after vaccination with OVA-CpG. As shown in FIGS. 16A-16B, Ifngr1-sgRNA cells had a slight competitive advantage among T cells. Additional experiments demonstrate the presence of a strong (FIGS. 18A-18B) or moderate (FIGS. 17A-17B) competitive advantage for Ifngr1-deficient CD8 T cells in isotype (antibody)-treated mice and equal ratios of control and Ifngr1-deficient T cells in mice treated with an anti-PD1 antibody. The plots of FIGS. 17A-17B also indicate that there was more enrichment of Ifngr1-KO cells 1 week after tumor implant in dLNs, relative to the vaccine-treated cells. No difference in the Tcf7 or CD44/CD62L phenotypes was observed. Furthermore, FIGS. 19A-19B demonstrates the alteration of T cell memory phenotypes in Ifngr1-deficient T cells compared to control CD8+ T cells in isotype-treated mice. The results shown in FIGS. 18A-18B and 19A-19B indicate some reversal of T cell enrichment follow anti-PDL1 antibody treatment.

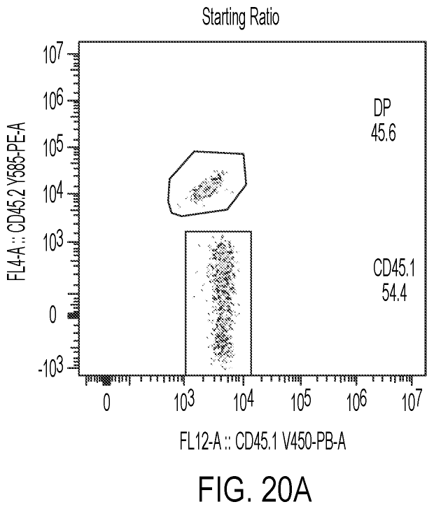

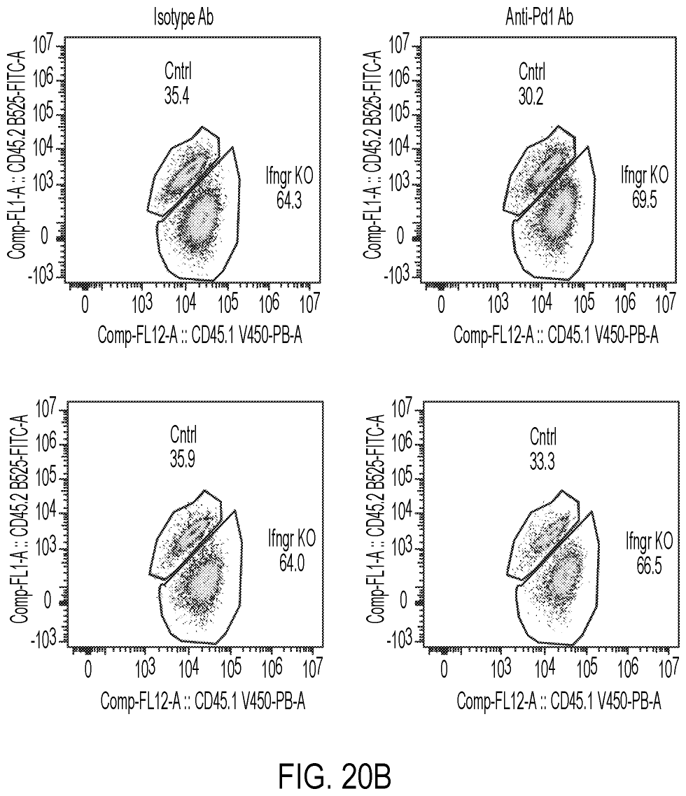

A slight enrichment in Ifngr1-sgRNA cells in isotype-treated animals and further enrichment in anti-PD1 treated animals was observed, in FIGS. 20A-20B. This data suggests there is increased potential for either recruitment of new T cells or T cell expansion in Ifngr1-deficient relative to control T cells.

To assess whether Ifngr1 deletion leads to better T cell persistence in vivo, control or Ifngr1-sgRNAs were delivered to naïve CD8 T cells isolated from Cas9/OT1-double transgenic mice. The cells were then expanded in vitro with anti-CD3/anti-CD28 stimulation for 1 week, then transferred to new recipient mice that had already had 1×10⁶ MC38-OVA tumors implanted 1 week prior to T cell transfer. Various numbers of T cells were transferred to recipient mice, with the goal of finding conditions that led to complete tumor clearance (neutralization or cytotoxicity).

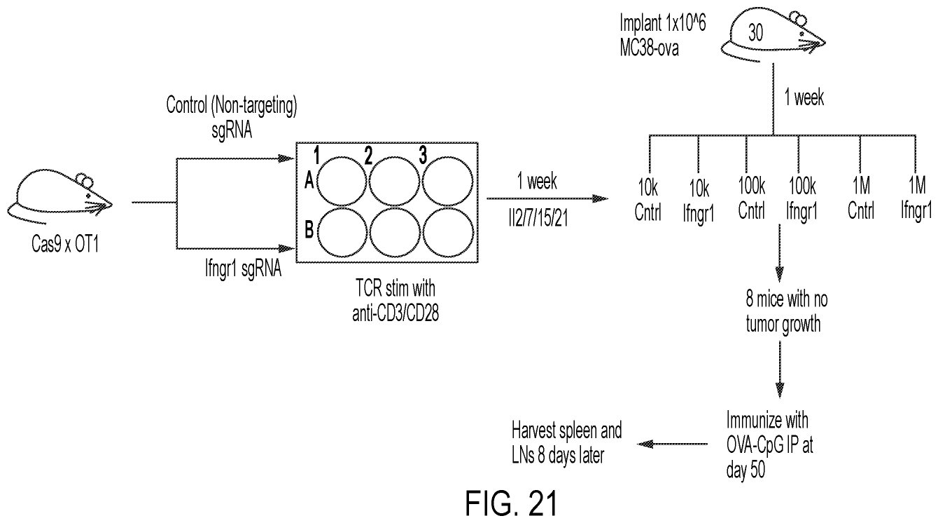

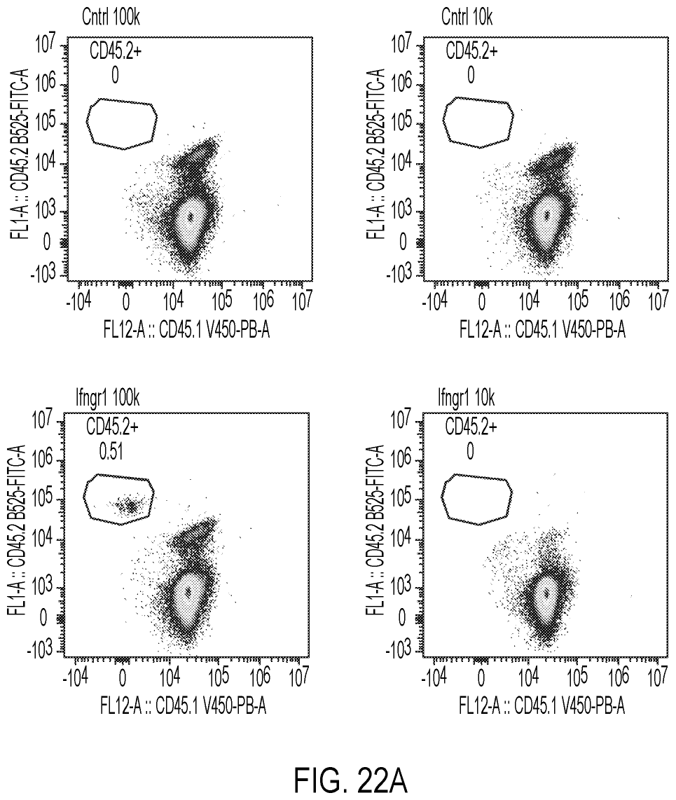

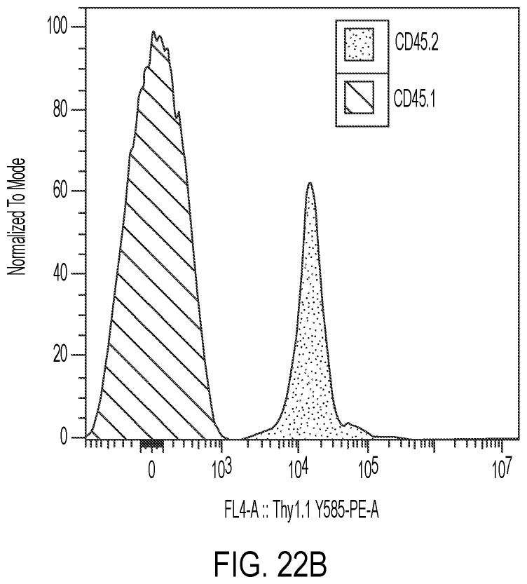

As shown in FIG. 21, there were 8 recipient mice that completely cleared their tumors, and those mice were immunized with OVA-CpG 50 days after T cell transfer. Eight days after vaccination, spleens and lymph nodes were harvested to quantify the number of transferred OT1 T cells that persisted. As shown in FIGS. 22A-22B, the use of congenic markers CD45.1 and CD45.2, transferred OT1 cells were only detected in mice that received Ifngr1-sgRNA containing CD8 T cells.

This data suggests that interferon-γ promotes differentiation and Tcf7 silencing in CD8 T cells following CD3/CD28 stimulation and decreases survival of CD8 T cells in tumor co-cultures. This suggests that Ifngr1-deficiency increases longevity and persistence of tumor-reactive CD8 T cells.

Subsequently, adoptive cell therapy experiments are performed to evaluate the expansion of OT1 T cells in vitro after inhibition by i) sgRNA or ii) antibody-mediated inhibition of the Ifngr1 gene compared to control, prior to transfer of these T cells to mice having tumors. As an initial step, control vs Ifngr1 knockout (KO) naïve CD8 T cells were transferred to mice with MC38-OVA tumors. It is hypothesized based on the above-described results that Ifngr1 knockout promotes persistence and anti-tumor activity of these cells.

A prospective method to assess longer-term persistence involves the transfer of a mixture of control and Ifngr1 KO OT1 T cells to MC38-OVA tumors followed by a rechallenge with B16-OVA later and measure competitive survival. Another prospective method involves reversible inhibition by expanding OT1 T cells with isotype or anti-Ifngr and anti-Ifng antibodies, followed by a transfer of these T cells to mice with B16-OVA tumors at a timepoint (i.e., 8-10 days) when the Tcf7 phenotype is strongly divergent among T cells.

### Example 3: In Vivo Screens to Investigate Other Tcf7 Regulators in Tumors

Perturb-seq (CRISP-seq) experiments were performed in Cas9/OT1 double transgenic mice in vivo in conjunction with analysis of CRISPR screens using single cell RNA sequencing (RNA-seq). Perturb-seq is a reverse genetics approach that allows for the investigation of phenotypes at the level of the transcriptome in a massively parallel fashion, by applying genetic perturbations to knock down a gene and study the resulting phenotype. As with the experiments of Example 1, a reversible perturbation method is applied for transient reduction of inhibitors of Tscm by CRISPR.

Perturb-seq experiments have been technically challenging in the context of T cells in vivo. A recent study has established novel techniques for direct capture of sgRNAs in single cell experiments. See Replogle et al., Nat Biotechnol. 2020 August; 38(8): 954-961, which is incorporated herein by reference. The methods in Replogle et al. were adopted to CD8+ T cells using naïve T cell transduction methods with two lentiviral vectors, as described in the experiments of Example 1. However, the protocol was modified by designing nested amplification primers to specifically amplify sgRNA sequences from single cell RNA sequencing experiments.

These experiments involve the following steps: i) transduction of two different lentiviral sgRNA libraries into naïve CD8+ T cells from Cas9/OT1 double transgenic mice, ii) transfer of these transduced cells into congenically marked (CD45.1 and CD45.2) recipient mice, iii) implantation of B16-OVA tumor cells the day after T cell transfer, and iv) isolation of all transferred or transduced CD8+ T cells from tumors 2 weeks after tumor implantation.

After a purification of live, transduced CD8+ T cells isolated from tumor tissues, the T cells are loaded onto single-cell sequencing chips (10× Genomics) and processed using 5′ V2 kits using the modified protocol described above to amplify sgRNA sequences in addition to the full transcriptome.

The data from these experiments is analyzed and will show the effect of perturbing newly identified Tcf7 regulators in tumor-reactive T cells in vivo. This will demonstrate the effects on altering T cell differentiation within tumors and characterizing the full transcriptome effect of each perturbation in individual cells.

The candidate modulator genes evaluated in parallel in the above-described in vivo screens are listed in Table 1, below. The first column lists a pool of 70 genes designed to initially test lentiviral transduction in T cells. These genes are hypothesized to be important Tcf7 regulators based on data from human biopsies comparing transcriptional and epigenetic differences between Tcf7-positive and Tcf7-negative T cells, and existing literature. The second column lists a pool of 100 genes. These represent 100 of the top scoring genes in the in vitro whole genome screen described in Example 1 (see FIGS. 7A-7C). Of these 100, 75 are from the ‘right’ side of the volcano plot of FIG. 7B, which are hypothesized to be negative regulators of Tcf7, and 25 from the ‘left’ side of the volcano plot that are hypothesized to be positive regulators of Tcf7. IFNGR1, JAK2, and STAT1 are among these 100 genes.

The third column of Table 1 lists seven genes that are present in both the 100 gene and 70 gene in vivo pools, which are expected to have strong effects on T cell state and Tcf7 expression, and as such will represent controls to compare the two in vivo screens. Tcf7 is among these genes.

TABLE 1

| | | | | | | | Shared Genes Between |
|---|---|---|---|---|---|---|---|
| 70 Gene Original Pool | | | 100 Gene Secondary Pool | | | | the In Vivo Pools |
| Arid4b | Gsk3b | Rad21 | Actb | Hif1a | Ndufs2 | Samd1 | Foxo1 |
| Arid5b | Hif1a | Rela | Ap1m1 | Hira | Nelfb | Slc2a1 | Hif1a |
| Atf2 | Hmgb1 | Runx2 | Arhgap1 | Hnrnpab | Pabpc1 | Slc35a2 | Myb |
| Bach2 | Hmgb2 | Runx3 | Arnt | Ifngr1 | Pgd | Slc7a1 | Prdm1 |
| Batf | Id2 | Satb1 | Atxn713 | Ifngr2 | Pgk1 | Slc7a5 | Runx3 |
| Bhlhe40 | Id3 | Sox4 | Bap1 | Il21r | Pgm3 | Smarca4 | Stat3 |
| Crem | Ikzf3 | Sp100 | Brd4 | Il2ra | Phip | Smarce1 | Tcf7 |
| Ctnnb1 | Il12rb1 | Sp140 | Ccdc6 | Ints5 | Pik3cd | Socs3 | |
| Dkk3 | Il12rb2 | Stat3 | Chd4 | Ints6 | Pik3cg | Spcs2 | |
| Dvl1 | Irf2 | Stat4 | Cited2 | Irf4 | Pik3r5 | Spcs3 | |
| Dvl2 | Irf9 | Sub1 | Cox7b | Itk | Pkm | Stat1 | |
| Dvl3 | Klf2 | Tbx21 | Ctcf | Jak2 | Ppp2r2a | Stat3 | |
| Egr1 | Ldhb | Tcf3 | Dcun1d3 | Lck | Prdm1 | Stk11 | |
| Elf1 | Lef1 | Tcf7 | Dnmt1 | Ldha | Pten | Sumo2 | |
| Eomes | Litaf | Tox | Dnttip1 | Mapk14 | Ptpn1 | Tcf7 | |
| Ep300 | Lrp1 | Tox2 | Drosha | Mbd2 | Ptpn2 | Tfap4 | |
| Ets1 | Mafk | Tpt1 | Dyrk1a | Med13 | Ptpn6 | Tkt | |
| Ezh2 | Myb | Yy1 | Elovl1 | Med16 | Ptprc | Traf3 | |
| Fosb | Nr3c1 | Zeb2 | Emc6 | Mideas | Rack1 | Ube2m | |
| Foxm1 | Nr4a1 | Zfp292 | Fbxo42 | Mlst8 | Rbbp4 | Uhrf1 | |
| Foxo1 | Nr4a2 | | Foxo1 | Mob4 | Rc3h1 | Umps | |
| Foxp1 | Nr4a3 | | Gale | Myb | Rpl28 | Vhl | |
| Fzd1 | Oxnad1 | | Glrx5 | Naa20 | Rpl28−ps1 | Washc4 | |
| Fzd3 | P2rx7 | | Gmppb | Ndrg3 | Rtf2 | Zc3h12a | |
| Fzd6 | Prdm1 | | Gpi1 | Ndufa6 | Runx3 | Zfp384 | |

Table 2, below, shows 239 candidate modulator genes, which include the 170 unique genes listed in Table 1, that have p values below 10⁻³, which is used as a criterion for additional screens and validation experiments. The numbers in the column to the left of each gene represent the log-fold change: if the number is positive the gene is a possible negative regulator of Tcf7, while if the number is negative the gene is a possible positive regulator of Tcf7.

TABLE 2
All genes in whole genome screen below p-value of 10\hat{ }−3

| | | | | | | | | | | | | | |
|---|---|---|---|---|---|---|---|---|---|---|---|---|---|
| 9 | Arnt | 5 | Atxn713 | 4 | Rtf2 | 4 | Drosha | 3 | Srp19 | 3 | Dpagt1 | 3 | Fh1 |
| −12 | Pten | 5 | Ifngr2 | 4 | Pgk1 | −3 | Uba3 | −3 | Srsf6 | 3 | Senp3 | 3 | Srp9 |
| −8 | Foxo1 | 6 | Runx3 | −3 | Sbno2 | 4 | Rpl28 | 3 | Thoc3 | 3 | Uqcrc1 | 3 | Pdcl |
| −8 | Myb | 5 | Pik3cg | 4 | Hnrnpab | 4 | Rpl28-ps1 | 3 | Irf2 | 3 | Kif20a | 3 | Jak1 |
| 7 | Prdm1 | 5 | Samd1 | 4 | Gale | −3 | Nrf1 | 3 | Slc19a1 | 3 | Gadd45g | 3 | Nus1 |
| −7 | Ube2m | 5 | Mapk14 | 4 | Slc2a1 | −3 | Smad4 | 3 | Grb2 | −4 | Ddx6 | | |
| 7 | Hif1a | −5 | Zc3h12a | −4 | Pdcd5 | 3 | Aldoa | −3 | Csde1 | −2 | Wdr26 | | |
| −7 | Ptpn2 | 5 | Traf3 | 4 | Glrx5 | −3 | Epc2 | 3 | Phb | −3 | Grk2 | | |
| −6 | Mbd2 | −4 | Ube2f | −3 | Mybl1 | 4 | Il2ra | 3 | Il21r | 3 | Tet2 | | |
| −6 | Ptprc | −4 | Ints5 | 4 | Lck | 4 | Ndufs2 | −3 | Hif1an | −3 | Nedd8 | | |
| −10 | Tcf7 | −4 | Keap1 | −3 | Chic2 | 3 | Zbtb32 | 3 | Med24 | −3 | Scaf4 | | |
| −6 | Ptpn1 | 4 | Slc35a2 | −3 | Mta2 | 3 | Crtc2 | 3 | Gnb2 | 3 | Ugp2 | | |
| 6 | Fbxo42 | −4 | Kmt2a | 4 | Mob4 | 3 | Wdr20 | 3 | Il7r | −3 | Ndc1 | | |
| 7 | Spcs3 | 4 | Pgd | 4 | Cox7b | −3 | Rnf7 | −3 | Nup43 | 3 | Chd7 | | |
| −6 | Socs3 | −4 | Cul5 | −3 | Tada2b | 4 | Ap1ml | 3 | Mthfd11 | 3 | Zap70 | | |
| 6 | Dyrk1a | −4 | Cul3 | −3 | Ddx3x | 3 | Huwe1 | 3 | Lsm10 | −3 | Syncrip | | |
| −5 | Ints6 | −5 | Arhgap1 | −3 | Dek | −3 | Aip | 3 | Lsm11 | 3 | Ddx56 | | |
| 7 | Ctcf | −4 | Itpkb | 4 | Actb | −3 | Smg7 | 3 | Mrpl20 | 3 | Cabin1 | | |
| −5 | Dnmt1 | −4 | Sgf29 | 3 | Akt1 | 3 | Pgm3 | 3 | Id2 | −2 | Tmem127 | | |
| −5 | Phip | 4 | Naa20 | 4 | Ccdc6 | −3 | Arih2 | −3 | Ccnd3 | −3 | Ccdc130 | | |
| 6 | Ppp2r2a | 5 | Ifngr1 | 4 | Brd4 | 3 | Tkt | 3 | Krr1 | −2 | Mynn | | |
| −5 | Stk11 | −3 | Hdac7 | 3 | Pik3cd | 3 | Trim28 | 4 | Pabpc1 | 3 | Ythdf2 | | |
| 6 | Spcs2 | −5 | Rc3h1 | −3 | Birc6 | −3 | Foxp1 | −3 | Ascc3 | −3 | Nosip | | |
| 5 | Stat3 | 4 | Mlst8 | 4 | Sumo2 | 4 | Gmppb | 4 | Med16 | 3 | Xpo6 | | |
| −4 | Vhl | −4 | Zbtb7a | −4 | Cish | 3 | Rictor | 4 | Rack1 | 3 | Pgp | | |
| 6 | Ldha | −4 | Slc33a1 | −3 | Tbl1xr1 | −4 | Dgkq | 3 | Hsp90ab1 | −2 | Ppcs | | |
| −5 | Ptpn6 | −4 | Eloc | −4 | Socs1 | −4 | Dhx29 | 3 | Srm | 3 | Tlk2 | | |
| 6 | Stat1 | 5 | Rbbp4 | 4 | Ndufa6 | 4 | Elovl1 | 3 | Ccna2 | 3 | Zbtb1 | | |
| −5 | Cited2 | 5 | Gpi1 | 3 | Wdr82 | −3 | Spop | 3 | F8a | 3 | Nelfcd | | |
| 6 | Hira | 4 | Pik3r5 | 3 | Usp32 | 3 | Uba2 | 3 | Ndufa3 | 3 | Xpo1 | | |
| 5 | Mideas | −4 | Cdca3 | 4 | Tfap4 | 4 | Itk | 3 | Hcfc2 | 3 | B4galt7 | | |
| −5 | Zfp384 | −4 | Cdk13 | 3 | Slc7a1 | −3 | Smg9 | 3 | Slc7a5 | 3 | Ptbp1 | | |
| −4 | Med13 | −3 | Dnajc2 | 3 | Umps | −2 | Cd5 | 3 | Ost4 | 3 | Vps52 | | |
| −5 | Uhrf1 | 4 | Bap1 | −3 | Jade2 | 3 | Chd4 | 4 | Washc4 | −2 | Phf6 | | |
| −4 | Strap | 5 | Smarce1 | 4 | Irf4 | −3 | Strada | −2 | Mark2 | −2 | Ahctf1 | | |
| 5 | Pkm | 4 | Nelfb | −3 | Srsf5 | 3 | Supv3l1 | 3 | Sympk | 3 | Cd3d | | |
| 5 | Dnttip1 | −3 | Meaf6 | 4 | Emc6 | 3 | Klf13 | 3 | Isca1 | 3 | Ndrg3 | | |
| 5 | Jak2 | −3 | Ints8 | 4 | Smarca4 | 3 | Zranb2 | 3 | Get4 | 3 | Cd2 | | |
| −4 | Dcun1d3 | −4 | Taf51 | −3 | Psmd4 | 3 | Ambra1 | −3 | Bcl2 | −2 | Peli1 | | |

### REFERENCES

Abelin, Jennifer G., Derin B. Keskin, Siranush Sarkizova, Christina R. Hartigan, Wandi Zhang, John Sidney, Jonathan Stevens, et al. 2017. “Mass Spectrometry Profiling of HLA-Associated Peptidomes in Mono-Allelic Cells Enables More Accurate Epitope Prediction.” Immunity 46 (2): 315-26.
Baharom, Faezzah, Ramiro A. Ramirez-Valdez, Kennedy K. S. Tobin, Hidehiro Yamane, Charles-Antoine Dutertre, Ahad Khalilnezhad, Glennys V. Reynoso, et al. 2021. “Intravenous Nanoparticle Vaccination Generates Stem-like TCF1+Neoantigen-Specific CD8+ T Cells.” Nature Immunology 22 (1): 41-52.
Corsello, Steven M., Rohith T. Nagari, Ryan D. Spangler, Jordan Rossen, Mustafa Kocak, Jordan G. Bryan, Ranad Humeidi, et al. 2020. “Discovering the Anti-Cancer Potential of Non-Oncology Drugs by Systematic Viability Profiling.” Nature Cancer 1 (2): 235-48.
Hu, Zhuting, Annabelle J. Anandappa, Jing Sun, Jintaek Kim, Donna E. Leet, David J. Bozym, Christina Chen, et al. 2018. “A Cloning and Expression System to Probe T-Cell Receptor Specificity and Assess Functional Avidity to Neoantigens.” Blood 132 (18): 1911-21.
Keskin, Derin B., Annabelle J. Anandappa, Jing Sun, Itay Tirosh, Nathan D. Mathewson, Shuqiang Li, Giacomo Oliveira, et al. 2019. “Neoantigen Vaccine Generates Intratumoral T Cell Responses in Phase Ib Glioblastoma Trial.” Nature 565 (7738): 234-39.
Krishna, Sri, Frank J. Lowery, Amy R. Copeland, Erol Bahadiroglu, Ratnadeep Mukherjee, Li Jia, James T. Anibal, et al. 2020. “Stem-like CD8 T Cells Mediate Response of Adoptive Cell Immunotherapy against Human Cancer.” Science 370 (6522): 1328-34.
Kurtulus, Sema, Asaf Madi, Giulia Escobar, Max Klapholz, Jackson Nyman, Elena Christian, Mathias Pawlak, et al. 2019. “Checkpoint Blockade Immunotherapy Induces Dynamic Changes in PD-1-CD8+Tumor-Infiltrating T Cells.” Immunity 50 (1): 181-94.e6.
Lin, Wen-Hsuan W., Simone A. Nish, Bonnie Yen, Yen-Hua Chen, William C. Adams, Radomir Kratchmarov, Nyanza J. Rothman, Avinash Bhandoola, Hai-Hui Xue, and Steven L. Reiner. 2016. “CD8+T Lymphocyte Self-Renewal during Effector Cell Determination.” Cell Reports 17 (7): 1773-82.
Nguyen, David N., Theodore L. Roth, P. Jonathan Li, Peixin Amy Chen, Ryan Apathy, Murad R. Mamedov, Linda T. Vo, et al. 2020. “Polymer-Stabilized Cas9 Nanoparticles and Modified Repair Templates Increase Genome Editing Efficiency.” Nature Biotechnology 38 (1): 44-49.
Ott, Patrick A., Zhuting Hu, Derin B. Keskin, Sachet A. Shukla, Jing Sun, David J. Bozym, Wandi Zhang, et al. 2017. “An Immunogenic Personal Neoantigen Vaccine for Patients with Melanoma.” Nature 547 (7662): 217-21.
Park, Ryan J., Tim Wang, Dylan Koundakjian, Judd F. Hultquist, Pedro Lamothe-Molina, Blandine Monel, Kathrin Schumann, et al. 2017. “A Genome-Wide CRISPR Screen Identifies a Restricted Set of HIV Host Dependency Factors.” Nature Genetics 49 (2): 193-203.
Roth, Theodore L., P. Jonathan Li, Franziska Blaeschke, Jasper F. Nies, Ryan Apathy, Cody Mowery, Ruby Yu, et al. 2020. “Pooled Knockin Targeting for Genome Engineering of Cellular Immunotherapies.” Cell 181 (3): 728-44.e21.
Roth, Theodore L., Cristina Puig-Saus, Ruby Yu, Eric Shifrut, Julia Carnevale, P. Jonathan Li, Joseph Hiatt, et al. 2018. “Reprogramming Human T Cell Function and Specificity with Non-Viral Genome Targeting.” Nature 559 (7714): 405-9.
Sade-Feldman, M., K. Yizhak, S. L. Bjorgaard, J. P. Ray, C. G. de Boer, R. W. Jenkins, D. J. Lieb, et al. 2018. “Defining T Cell States Associated with Response to Checkpoint Immunotherapy in Melanoma.” Cell 175 (4): 998-1013 e20.
Sarkizova, Siranush, Susan Klaeger, Phuong M. Le, Letitia W. Li, Giacomo Oliveira, Hasmik Keshishian, Christina R. Hartigan, et al. 2020. “A Large Peptidome Dataset Improves HLA Class I Epitope Prediction across Most of the Human Population.” Nature Biotechnology 38 (2): 199-209.
Siddiqui, Imran, Karin Schaeuble, Vijaykumar Chennupati, Silvia A. Fuertes Marraco, Sandra Calderon-Copete, Daniela Pais Ferreira, Santiago J. Carmona, et al. 2019. “Intratumoral Tcf1+PD-1+CD8+ T Cells with Stem-like Properties Promote Tumor Control in Response to Vaccination and Checkpoint Blockade Immunotherapy.” Immunity 50 (1): 195-211.e10.

### Other Embodiments

The foregoing has been a description of certain non-limiting embodiments of the disclosure. Those of ordinary skill in the art will appreciate that various changes and modifications to this description may be made without departing from the spirit or scope of the present disclosure, as defined in the following claims.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the disclosure shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of preparing a modified T cell comprising:
i) isolating one or more naïve T cells;
ii) contacting a T cell with an agent that stimulates expansion of the T cell; and
iii) incubating the T cell with an inhibitor of a negative modulator gene of TCF7 for at least 18 hours.

2. The method of claim 1, wherein the T cell is incubated with the inhibitor for at least 24 hours, 30 hours, 36 hours, 48 hours, 54 hours, 72 hours, 4 days, or 5 days.

3. The method of claim 1 or 2, wherein the inhibitor is a small molecule inhibitor of the activity of the protein encoded by the negative modulator gene.

4. The method of claim 1 or 2, wherein the inhibitor is a short interfering RNA comprising a sequence of at least 10 contiguous nucleotides that is complementary to a portion of a genomic sequence comprising the negative modulator gene.

5. The method of claim 1 or 2, wherein the inhibitor is a complex comprising i) a CRISPR-associated protein, and ii) a guide RNA comprising a guide sequence of at least 10 contiguous nucleotides that is complementary to a portion of a genomic sequence comprising the negative modulator gene.

6. The method of claim 1 or 2, wherein the inhibitor is a polynucleotide encoding an siRNA molecule.

7. The method of claim 1 or 2, wherein the inhibitor is a polynucleotide encoding a complex comprising a CRISPR-associated protein and a guide RNA comprising a guide sequence of at least 10 contiguous nucleotides that is complementary to a portion of a genomic sequence comprising the negative modulator gene.

8. The method of claim 5 or 7, wherein the CRISPR-associated protein is a fusion protein comprising a dCas9 domain and a KRAB domain.

9. The method of any one of claims 1-8, wherein the candidate modulator gene is a gene that encodes a transcription factor.

10. The method of any one of claims 1-9, wherein the candidate modulator gene is in a gene selected from the group consisting of IFNGR1, JAK2, ARNT, STAT1, IRF1, TBX2, TFG, PPP2R2A, DCTN5, ASF1A, DDX1I, HIRA, SMC4, MRPL53, TRIML2, and DNAJC11.

11. The method of any one of claims 1-10, wherein the candidate modulator gene is the JAK2 gene or the STAT1 gene.

12. The method of any one of claims 1-10, wherein the candidate modulator gene is the IFNGR1 gene.

13. The method of any one of claims 1-12, wherein the method is performed in vitro or ex vivo.

14. A method of preparing a population of modified T cells comprising:
i) isolating a population of naïve T cells;
ii) contacting the population with an agent that stimulates expansion of the T cells; and
iii) incubating the T cells with an inhibitor of a negative modulator gene of TCF7 for at least 18 hours.

15. The method of claim 14, wherein the T cells exhibit a memory or a stem-cell memory (Tscm) phenotype after the step of incubating.

16. The method of any one of claims 1-15 further comprising:
iv) engineering the T cell or T cells to express an antigen-specific T-cell receptor (TCR) that is associated with a tumor-infiltrating lymphocyte (TIL) in a subject using homology directed repair.

17. The method of claim 16 further comprising:
v) contacting the T cell or T cells with one or more T cell costimulatory factors.

18. The method of any one of claims 1-17, wherein the inhibitor is a small molecule inhibitor of JAK2.

19. The method of any one of claims 1-18, wherein the inhibitor is selected from ruxolitinib, baricitinib, fedratinib, gandotinib, lestaurtinib, momelotinib, and pacritinib.

20. The method of any one of claims 1-19, wherein the inhibitor is a small molecule inhibitor of STAT.

21. The method of any one of claims 1-20, wherein the inhibitor is selected from pravastatin, ISS-840, fludarabine, and OPB-31121.

22. The method of any one of claims 1-21, wherein the step of isolating comprises isolating and/or purifying the T cells from the blood of a subject.

23. The method of any one of claims 1-22, wherein the step of isolating comprises isolating and/or purifying the T cells from the blood of a cancer patient.

24. A method of genetic screening comprising:
i) evaluating a first level of expression of TCF7 protein in a naïve T cell;
ii) contacting the T cell in vitro with a) a CRISPR-associated nuclease, and b) a guide RNA molecule that comprises a guide sequence of at least 10 contiguous nucleotides that is complementary to a target sequence in the genomic DNA of the T cell;
iii) contacting the cell with an agent that stimulates expansion of the T cell;
iv) evaluating a second level of expression of TCF7 protein in the T cell; and
v) identifying the target sequence as a candidate modulator gene of TCF7 if the second level of expression exceeds the first level by more than 10%.

25. A method of genetic screening comprising:
i) contacting a population of naïve T cells with a) a CRISPR-associated nuclease, and b) a library of guide RNA molecules, wherein each of the guide RNA molecules comprises a different guide sequence of at least 10 contiguous nucleotides that is complementary to a portion of a target sequence in the genomic DNA of a T cell;
ii) contacting the population with an agent that stimulates expansion of the T cells in the population;
iii) sorting the cells of the population based on level of expression of TCF7;
iv) evaluating the abundance of any of the plurality of guide RNA molecules in the cells exhibiting substantially higher expression of TCF7; and
v) identifying a target sequence as a candidate modulator gene if the abundance of the guide RNA molecule that is complementary to a portion of the candidate gene is substantially enriched in one or more cells exhibiting higher expression of TCF7.

26. The method of 24 or 25, wherein the step of contacting comprises transducing the one or more cells with one or more polynucleotides encoding the CRISPR-associated nuclease and the library of guide RNA molecules.

27. The method of 26, wherein the one or more polynucleotides is comprised within one or more lentiviral vectors.

28. The method of 27, wherein each of the lentiviral vectors is encapsulated in a lentiviral envelope.

29. The method of 28, wherein the lentiviral envelope comprises VSV-g and ecotropic envelope proteins.

30. The method of any one of claims 24-29, wherein the CRISPR-associated nuclease is a Cas9 nuclease.

31. The method of any one of claims 24-30, wherein each of the guide RNA molecules is a single-guide RNA (sgRNA) molecule.

32. The method of any one of claims 25-31, wherein the library comprises at least 10, at least 15, at least 20, at least 30, at least 40, at least 50, at least 60, at least 65, at least 70, at least 75, at least 80, at least 90, or at least 100 guide RNA molecules.

33. The method of any one of claims 24-32, wherein the candidate modulator gene encodes a transcription factor.

34. The method of any one of claims 24-33, wherein the candidate modulator gene is a gene selected from the group consisting of IFNGR1, JAK2, ARNT, STAT1, IRF1, TBX2, TFG, PPP2R2A, DCTN5, ASF1A, DDX1I, HIRA, SMC4, MRPL53, TRIML2, and DNAJC11.

35. The method of any one of claims 24-34, wherein the candidate modulator gene is JAK2 or STAT.

36. The method of any one of claims 24-34, wherein the candidate modulator gene is IFNGR1.

37. The method of any one of claims 24-36, wherein the agent that stimulates expansion of the T cell comprises a monoclonal anti-CD3 antibody and/or a monoclonal anti-CD28 antibody.

38. The method of any one of claims 25-37, wherein the step of sorting comprises flow cytometry-mediated sorting based on expression of a fluorescent reporter gene that is co-expressed with TCF7 in the cell.

39. The method of any one of claims 25-36 further comprising:
vi) administering to a tissue of interest in an experimental subject a population of T cells comprising i) a plurality of guide RNA molecules, wherein each of the guide RNA molecules comprises a different guide sequence of at least 10 contiguous nucleotides that is complementary to a portion of a candidate modulator gene, and ii) a CRISPR-associated protein;
vii) administering to the tissue of interest an agent that stimulates T cell expansion;
viii) evaluating the abundance of any of the plurality of guide RNA molecules in the tissue of interest at one or more time points; and
ix) identifying a candidate modulator gene as a negative modulator gene of TCF7 if the abundance of a guide RNA molecule comprising a guide sequence that is complementary to the candidate modulator gene is substantially enriched in the population over time.

40. The method of 39, wherein step viii) further comprises evaluating the degree of proliferation of one or more T cells in the tissue of interest.

41. The method of 39 or 40, wherein the CRISPR-associated protein comprises a nuclease-inactive Cas9 (dcas9) protein.

42. The method of any one of claims 39-41, wherein each of the guide molecules is a doxycycline-inducible sgRNA molecule.

43. The method of any one of claims 39-42, wherein the experimental subject is a rodent.

44. The method of any one of claims 39-43, wherein the tissue of interest is tumor tissue or lymphatic tissue.

45. The method of any one of claims 39-44, wherein the tissue of interest is tumor tissue.

46. The method of any one of claims 39-45, wherein the agent that stimulates T cell expansion is a vaccine.

47. The method of any one of claims 44-46, wherein step ix) further comprises evaluating changes in size of the tumor tissue over time.

48. The method of any one of claims 39-47, wherein the plurality comprises at least 10, at least 15, at least 20, at least 30, at least 35, at least 40, at least 45, or at least 50 guide RNA molecules.

49. The method of any one of claims 39-48, wherein the CRISPR-associated protein comprises a fusion of a dCas9 protein and a transcription factor.

50. The method of any one of claims 44-49, wherein the cells in the tumorigenic tissue exhibit surface expression of ovalbumin (OVA) antigen.

51. The method of any one of claims 24-50, wherein the method is performed in vitro or ex vivo.

52. The method of any one of claims 49-51, wherein the transcription factor is a KRAB zinc finger protein.

53. A method of genetic screening comprising:
contacting a population of naïve T cells with one or more lentiviral particles containing a recombinant vector comprising polynucleotides encoding a) a CRISPR-associated nuclease, and b) a plurality of guide RNA molecules, wherein each of the guide RNA molecules comprises a different guide sequence of at least 10 contiguous nucleotides that is complementary to a target sequence in the genomic DNA of a T cell,
whereby the population of cells are transduced with the plurality of guide RNA molecules,
wherein the one or more lentiviral particles comprise VSV-g and ecotropic envelope proteins.

54. The method of 53, wherein at least 60%, at least 65%, at least 70%, at least 75%, at least 80%, at least 85%, at least 90%, at least 95%, or at least 99% of the T cells are viable 72 hours after contacting with the one or more lentiviral particles.

55. The method of 53 or 54, wherein the T cells exhibit a memory or a stem-cell memory (Tscm) phenotype after the step of contacting.

56. The method of any one of claims 53-55, wherein the method is performed in vitro or ex vivo.

57. The method of any one of claims 53-56 further comprising contacting the population with one or more the reagents selected from RetroNectin*, LentiBOOST P®, recombinant IL7, and recombinant IL15.

58. A lentiviral particle comprising a recombinant lentiviral vector comprising one or more polynucleotides encoding a) a CRISPR-associated nuclease, and b) a plurality of guide RNA molecules, wherein each of the guide RNA molecules comprises a different guide sequence of at least 10 contiguous nucleotides that is complementary to a target sequence in the genomic DNA of a T cell, and wherein the lentiviral particle comprises VSV-g and envelope proteins.

59. A composition comprising one or more modified T cells prepared according to any one of the methods of claims 1-23.

60. The composition of claim 59, wherein the T cells are polyclonal T cells.

61. The composition of claim 60, wherein the polyclonal T cells express a T-cell receptor (TCR) that is associated with a tumor-infiltrating lymphocyte (TIL).

62. The composition of any one of claims 59-61, wherein the T cells exhibit a memory or a stem-cell memory (Tscm) phenotype.

63. The composition of any one of claims 59-61 further comprising a pharmaceutically acceptable excipient.

64. The composition of any one of claims 59-63, wherein the composition is suitable for adoptive transfer to a subject.

65. The composition of any one of claims 59-63, wherein the composition is suitable for non-adoptive therapies.

66. The composition of 64, wherein the composition is adapted for autologous transfer to a subject.

67. The composition of any one of claims 59-66 for use in treating cancer.

68. A method of treating a subject suffering from, or diagnosed with, a cancer comprising administering the composition of any one of claims 59-67.

69. The method of claim 68, wherein the subject is a human.

70. A method of treating a subject suffering from, or diagnosed with, a cancer comprising:
i) isolating T cells from the blood of a subject;
ii) contacting the T cells ex vivo with an inhibitor of a negative modulator gene of TCF7 for at least 18 hours, thereby producing modified T cells; and
iii) administering the modified T cells to the subject.

71. A method of treating a subject suffering from, or diagnosed with, a cancer comprising:
i) isolating T cells from the blood of a first subject;
ii) contacting the T cells ex vivo with an inhibitor of a negative modulator gene of TCF7 for at least 18 hours, thereby producing modified T cells; and
iii) administering the modified T cells to a second subject.

72. The method of claim 71, wherein the T cells express a T-cell receptor (TCR) that is associated with a tumor-infiltrating lymphocyte (TIL).

73. The method of any one of claims 68-72, wherein the cancer is a solid tumor.

74. The method of any one of claims 68-72, wherein the cancer is a lymphoma or leukemia.

75. The method of any one of claims 68-74 further comprising administering a chemotherapeutic agent to the subject.

76. The method of any one of claims 68-75, wherein the modified T cells exhibit a Tscm phenotype and/or TCF7 overexpression.

77. The method of any one of claims 68-75, wherein the modified T cells exhibit are cytolytic T cells, NK T cells, and/or CD8+ T cells.

78. The method of any one of claims 68-77, wherein the step of contacting comprises incubating the T cells with the inhibitor for at least 24 hours, 30 hours, 36 hours, 48 hours, 54 hours, 72 hours, 4 days, or 5 days.

79. The method of any one of claims 68-78, wherein the isolating step further comprises contacting the T cells with an agent that stimulates expansion of the T cells.

80. The method of claim 79, wherein the agent that stimulates T cell expansion is a vaccine.

81. The method of claim 79 or 81, wherein the agent that stimulates T cell expansion is an OVA-CpG vaccine.
